(19) United States
(12) Patent Application Publication
(10) Pub. No.: US 2026/0249096 A1
(43) Pub. Date: Aug. 27, 2026

(54) SYSTEMS AND METHODS FOR TREATING EYELID INFLAMMATION

(71) Applicant: The General Hospital Corporation, Boston, MA (US)

(72) Inventors: Richard R. Anderson, Boston, MA (US); Tianhong Dai, Boston, MA (US); Yakir Levin, Boston, MA (US)

(21) Appl. No.: 18/872,760

(22) PCT Filed: Jun. 7, 2023
(86) PCT No.: PCT/US2023/068096

§ 371 Date: Dec. 6, 2024

Related U.S. Application Data
(60) Provisional application No. 63/349,746, filed on Jun. 7, 2022.

(51) Int. Cl. A61N 5/06

(52) U.S. Cl. CPC A61N 5/0603; A61N 5/0613; A61N 2005/0659; A61N 2005/0663

(57) ABSTRACT

An eyelid treatment system can include a contact lens that can include an inner concave surface and an outer convex surface opposite the inner concave surface. The contact lens can be configured to be placed on an eyeball of a subject. The eyelid treatment system can include a light source optically coupled to the contact lens. The light source can be configured to deliver light to an inner surface of an eyelid of the subject when the contact lens is placed on the eyeball. The inner concave surface can be configured to at least partially block light from being transmitted through the inner concave surface, such that the light propagates in a direction away from the eyeball of the subject. The light from the light source can be configured to reduce eyelid inflammation.

### CROSS-REFERENCE TO RELATED APPLICATIONS

This application claims priority to U.S. Patent Application No. 63/349,746 filed Jun. 7, 2022, and entitled, “Optical Contact Lens for the Treatment of Blepharitis,” which is hereby incorporated by reference in its entirety.

### STATEMENT REGARDING FEDERALLY SPONSORED RESEARCH

N/A

### BACKGROUND

Blepharitis, an inflammation of the eyelids, can present a wide range of symptoms including red eyes, watering eyes, itchy eyelids, eyelids that appear greasy, swollen and red eyelids, an increased need to blink, blurred vision, sensitivity to light, etc. Blepharitis negatively impacts nearly 25 million Americans with chronic Blepharitis appearing more often than acute Blepharitis. The exact cause of Blepharitis is currently unknown, but Blepharitis is frequently associated with atopic dermatitis, rosacea, demodicosis, seborrheic dermatitis, infection, allergies, etc.

Typical treatments of Blepharitis have been largely unsuccessful. Thus, it would be desirable to have improved systems and methods for treating eyelid inflammation.

### SUMMARY OF THE DISCLOSURE

Some non-limiting examples of the disclosure provide an eyelid treatment system that can include a contact lens that can include an inner concave surface and an outer convex surface opposite the inner concave surface. The contact lens can be configured to be placed on an eyeball of a subject. The eyelid treatment system can include a light source optically coupled to the contact lens. The light source can be configured to deliver light to an inner surface of an eyelid of the subject when the contact lens is placed on the eyeball. The inner concave surface can be configured to at least partially block light from being transmitted through the inner concave surface, such that the light propagates in a direction away from the eyeball of the subject. The light from the light source can be configured to reduce eyelid inflammation.

In some non-limiting examples, an inner concave surface can include an opaque material.

In some non-limiting examples, an inner concave surface can be positioned on a first side of a contact lens and a second side of the contact lens. An optical axis of the contact lens can be positioned between the first side of the contact lens and the second side of the contact lens.

In some non-limiting examples, an optical axis of the contact lens can intersect with an inner concave surface.

In some non-limiting examples, a contact lens can include an opaque material that can define an inner concave surface of the contact lens, such that light from a light source or ambient light that is transmitted through the outer convex surface towards the inner concave surface can be at least partially blocked from being transmitted therethrough. A contact lens can include a reflective material that can define the inner concave surface of the contact lens, such that the light from the light source or the ambient light that is transmitted through the outer convex surface towards the inner concave surface is at least partially blocked from being transmitted therethrough.

In some non-limiting examples, an eyelid treatment system can include a stem coupled to a contact lens. The stem can extend away from the contact lens in a direction away from an eyeball when the contact lens is placed on the eyeball.

In some non-limiting examples, a light source can be optically coupled to a stem, such that light from the light source can be transmitted through the stem and to a contact lens.

In some non-limiting examples, light from a light source can have a wavelength that is within a first range of 400-470 nanometers, such that the light is configured to destroy microbes of the eyelid. The light can have a wavelength that is within a second range of 620-750 nanometers, such that the light is configured to at least one of heat eyelid tissue or to decrease inflammation or calm the immune system of the eyelid tissue through photobiomodulation. The light can have a wavelength within a third range of 800-1100 nanometers, such that the light is configured to at least one of heat the eyelid tissue or to decrease inflammation or calm the immune system of the eyelid tissue through photobiomodulation.

In some non-limiting examples, light can have a first wavelength and a second wavelength. The first wavelength can be within a first, second, or third range. The second wavelength can be within the other of the first, second, or third range.

In some non-limiting examples, a contact lens can include a biocompatible material. The biocompatible material can be at least one of a hydrogel, silicone, a polymer, or a plastic.

In some non-limiting examples, a light source can be coupled to a contact lens.

In some non-limiting examples, an eyelid treatment system can include a power source coupled to the contact lens. A light source can be electrically coupled to the power source.

In some non-limiting examples, light (e.g., therapeutic light) from the light source (or emitted by a substrate) can be directed at, at least one of 25% of an inner surface area of the eyelid, 50% of the inner surface area of the eyelid, or 75% of the inner surface area of the eyelid.

In some non-limiting examples, a contact lens can be a diverging lens, such that light from a light source diverges when the light is emitted out from a outer convex surface of the contact lens.

Some non-limiting examples of the disclosure provide an eyelid treatment system. The eyelid treatment system can include a substrate that can include an inner concave surface and an outer convex surface opposite the inner concave surface. The substrate can be configured to be placed external to an eye of a subject. The outer concave surface can be configured to at least partially block ambient light from being transmitted through the outer convex surface in a direction towards the inner concave surface. The eyelid treatment system can include a light source that can be configured to deliver light to an outer surface of an eyelid of the subject when the substrate is placed over the eyelid of the subject. The light from the light source can be configured to improve eyelid inflammation.

In some non-limiting examples, an outer convex surface can include an opaque material.

In some non-limiting examples, an outer convex surface can be positioned on a first side of a substrate and a second side of the substrate. An axis of a substrate that bisects the substrate into the first side and the second side can be positioned between the first side of the substrate and the second side of the substrate.

In some non-limiting examples, a substrate can include an opaque material that can define an outer convex surface of the substrate, such that ambient light can be at least partially blocked from being transmitted through the outer convex surface of the substrate. The substrate can include a reflective material that can define the outer convex surface of the substrate, such that the ambient light can be at least partially blocked from being transmitted through the outer convex surface of the substrate.

In some non-limiting examples, an eyelid treatment system can include a stem coupled to a substrate. The stem can extend away from the substrate in a direction away from the eyelid when the substrate is placed over the eyelid.

In some non-limiting examples, a light source can be optically coupled to a stem, such that the light from the light source can be transmitted through the stem and to the substrate.

In some non-limiting examples, light from a light source can have a wavelength that is within a first range of 400-470 nanometers, such that the light is configured to destroy microbes of the eyelid. The light can have a wavelength that is within a second range of 620-750 nanometers, such that the light is configured to at least one of heat the eyelid tissue or to decrease inflammation or calm the immune system of the eyelid tissue through photobiomodulation. The light can have a wavelength that is within a third range of 800-1100 nanometers, such that the light is configured to at least one of heat the eyelid tissue or to decrease inflammation or calm the immune system of the eyelid tissue through photobiomodulation.

In some non-limiting examples, light can have a first wavelength and a second wavelength. The first wavelength can be within the first, second, or third range. The second wavelength can be within the other of the first, second, or third range.

In some non-limiting examples, a substrate can include a biocompatible material. A biocompatible material can be at least one of a hydrogel, silicone, a polymer, or a plastic.

In some non-limiting examples, a light source can be coupled to the substrate.

In some non-limiting examples, an eyelid treatment system can include a power source coupled to a substrate. A light source can be electrically coupled to the power source.

In some non-limiting examples, light (e.g., therapeutic light) from the light source (or emitted by a substrate) can be directed at, at least one of 25% of an outer surface area of the eyelid, 50% of the outer surface area of the eyelid, or 75% of the outer surface area of the eyelid.

In some non-limiting examples, a substrate can be a converging lens, such that light from a light source converges when the light is emitted out from the inner concave surface of the substrate.

Some non-limiting examples of the disclosure provide an eyelid treatment system. The eyelid treatment system can include a first substrate that can include a first inner concave surface and a first outer convex surface opposite the first inner concave surface. The first substrate can be configured to be placed on an eye of a subject. The first inner concave surface can be configured to at least partially block light from being transmitted through the inner concave surface away from the outer convex surface. The eyelid treatment system can include a second substrate that can include a second inner concave surface and a second outer convex surface opposite the second inner concave surface The second substrate can be configured to be placed external to the eye of the subject. The second outer convex surface can be configured to at least partially block ambient light from being transmitted through the second outer convex surface in a direction towards the second inner concave surface. The eyelid treatment system can include one or more light sources can be configured to emit light to at least one of an inner surface or an outer surface of the eyelid of the subject when the first substrate is placed on the eye of the subject and when the second substrate is placed over the eyelid of the subject. The light from the one or more light sources can be configured to reduce eyelid inflammation.

In some non-limiting examples, an eyelid treatment system can include a first stem coupled to a first substrate and extending away from the first substrate in a direction away from the eye of the subject.

In some non-limiting examples, a second substrate can include an aperture. A first stem can be configured to be inserted into the aperture of the second substrate.

In some non-limiting examples, an eyelid treatment system can include a second stem coupled to a second substrate and extending away from the second substrate in a direction away from the eye of the subject.

In some non-limiting examples, one or more light sources can include a first light source coupled to a first outer convex surface of a first substrate. The first light source can be configured to deliver a first light to an inner surface of an eyelid. The one or more light sources can include a second light source coupled to a second inner concave surface of a second substrate. The second light source can be configured to deliver a second light to an outer surface of the eyelid.

In some non-limiting examples, a first substrate can be a first lens and a second substrate can be a second lens. One or more light sources can be optically coupled to the first lens or the second lens.

In some non-limiting examples, a first substrate and a second substrate can be configured to compress an eyelid when the eyelid is placed between the first substrate and the second substrate to express contents of a Meibomian gland.

In some non-limiting examples, an eyelid treatment system can include an actuator that can be configured to move a first substrate relative to a second substrate or vice versa.

In some non-limiting examples, an eyelid treatment system can include a return spring coupled between a first substrate and a second substrate.

In some non-limiting examples, an eyelid treatment system a first substrate can include a protrusion extending away from a first outer convex surface and away from an eye. A second substrate can include a second protrusion extending away from a second inner concave surface and towards the eye.

Some non-limiting examples of the disclosure provide a method of treating eyelid inflammation. The method can include placing a substrate on an eye of a subject or over an eyelid of the subject and causing one or more light sources coupled to the substrate or optically coupled to the substrate to emit first light towards the eyelid or towards the substrate. The method can include reducing eyelid inflammation from the first light by the first light emitted from the one or more light sources and directed towards the eyelid causing a therapeutic effect in the eyelid tissue that decreases eyelid inflammation of the eyelid. The method can include reducing eyelid inflammation from the first light by the first light emitted from the one or more light sources and directed towards the substrate causes the substrate to emit a second light different from the first light towards the eyelid. The second light can cause a therapeutic effect in the eyelid tissue that decreases eyelid inflammation of the eyelid.

In some non-limiting examples, first light can have a plurality of different wavelengths of light. The first light emitted from one or more light sources and directed towards an eyelid can cause a plurality of different therapeutic effects in the eyelid tissue from the plurality of different wavelengths of light.

In some non-limiting examples, a second light can have a plurality of different wavelengths of light. The second light emitted by the substrate and directed towards the eyelid can cause a plurality of different therapeutic effects in the eyelid tissue from the plurality of different wavelengths of light.

In some non-limiting examples, a substrate can be a first substrate that can be optically coupled to one or more light sources. A method can include placing the first substrate on an eye of the subject, placing a second substrate over the eyelid of the subject, and causing the one or more light sources to deliver the first light to the first substrate and the second substrate thereby delivering the first light to opposing sides of the eyelid. The opposing sides of the eyelid can be an inner side and an outer side of the eyelid.

In some non-limiting examples, a substrate can be a first substrate. A first light source of one or more light sources can be coupled to the first substrate. A therapeutic effect can be a first therapeutic effect. A method can include placing the first substrate on an eye of a subject, and placing a second substrate over an eyelid of the subject. The one or more light sources can include a second light source coupled to the second substrate. The method can include causing the first light source to deliver first light to an inner side eyelid thereby causing the first therapeutic effect in the eyelid tissue that decreases eyelid inflammation of the eyelid. The method can include causing the second light source to deliver third light to the eyelid thereby causing a second therapeutic effect in the eyelid tissue that decreases eyelid inflammation of the eyelid.

In some non-limiting examples, a substrate can include a photoluminescent material. First light can be emitted towards the substrate that can excite the photoluminescent material, such that the photoluminescent material emits second light having a wavelength longer than the first light.

In some non-limiting examples, a therapeutic effect can include destroying microbes of an eyelid, improving an immune response of eyelid tissue, improving the blood flow of the eyelid tissue, or causing photobiomodulation of the eyelid tissue.

In some non-limiting examples, a first light or a second light can be blue light, violet light, red light, or near infrared light.

In some non-limiting examples, a substrate can be a first substrate. A method can include placing the first substrate on an eye of a subject, placing a second substrate over the eyelid of the subject after placing the first substrate on the eye of the subject and after the subject closes the eyelid, and advancing the first substrate towards the second substrate or advancing the second substrate towards the first substrate to express the Meibomian glands of the eyelid.

In some non-limiting examples, advancing a first substrate towards a second substrate or advancing the second substrate towards the first substrate includes translating, using an actuator, the first substrate towards the second substrate or the second substrate towards the first substrate.

In some non-limiting examples, a method can include moving a first substrate away from a second substrate or moving the second substrate away from the first substrate.

In some non-limiting examples, moving a first substrate or moving a second substrate can include unloading a spring coupled between the first substrate and the second substrate.

Some non-limiting examples of the disclosure provide an eyelid treatment system. The eyelid treatment system can include a substrate that can be configured to be placed on an eye of a subject, or over an eyelid of an eye of the subject. The eyelid treatment system can include a light source coupled to the substrate or optically coupled to the substrate. The light source can be configured to emit first light towards the eyelid or towards the substrate to reduce eyelid inflammation of the eyelid by the first light being directed towards the eyelid tissue that causes a therapeutic effect in the eyelid tissue that decreases eyelid inflammation of the eyelid, or the first light directed towards the substrate causes the substrate to emit a second light different from the first light towards the eyelid, the second light can cause a therapeutic effect in the eyelid tissue that decreases eyelid inflammation of the eyelid.

In some non-limiting examples, a substrate can include a photoluminescent material. First light can be emitted towards the substrate that can excite the photoluminescent material, such that the photoluminescent material emits second light having a wavelength longer than the first light.

Some non-limiting examples of the disclosure provide a device comprising an optical contact lens formed from compatible, light-transmitting and/or light-emitting materials and which delivers multiple wavelengths of therapeutic light to the eye lids for the treatment of blepharitis with minimal or no exposure to the retina and cornea, in accordance with any non-limiting example disclosed herein.

Some non-limiting examples of the disclosure provide a method of forming a device or system in accordance with any of the non-limiting examples described herein.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 shows a schematic illustration of a side view of an eyelid treatment system.

FIG. 2A shows a schematic illustration of a side view of an eye device.

FIG. 2B shows a schematic illustration of a side view of another eye device.

FIG. 2C shows a schematic illustration of a top view of the eye device of FIG. 2B.

FIG. 2B shows a schematic illustration of a side view of another eye device.

FIG. 3 shows a schematic illustration of a side view of another eyelid treatment system.

FIG. 4 shows a schematic illustration of a side view of another eyelid treatment system.

FIG. 5A shows a schematic illustration of a side view of another eye device.

FIG. 5B shows a schematic illustration of a side view of another eye device.

FIG. 5C shows a schematic illustration of a top view of the eye device of FIG. 5B.

FIG. 5D shows a schematic illustration of a side view of another eye device.

FIG. 6 shows a schematic illustration of a top, cross-sectional view of another eyelid treatment system.

FIG. 7 shows a schematic illustration of a side view of another eye device.

FIG. 8 shows a schematic illustration of a top view of the eye device of FIG. 7.

FIG. 9 shows a schematic illustration of a side view of another eye device.

FIG. 10 shows a schematic illustration of a top view of the eye device of FIG. 9.

FIG. 11 shows a flowchart of a process for reducing eyelid inflammation in an eyelid of a subject.

FIG. 12 shows a schematic of various optical contact lenses in accordance with a non-limiting example of the disclosure.

### DETAILED DESCRIPTION OF THE PRESENT DISCLOSURE

As described above, Blepharitis, an inflammation of the eyelids (e.g., which can also cause an inflammation of the eye under the eyelid), is a common eye disorder that negatively impacts many individuals around the world. Since the root cause of Blepharitis has been difficult to discern, it is not surprising that typical treatments for Blepharitis have been largely ineffective. These typical treatments include applying topical antibiotics, topical steroids, thermal heating of the eyelids, and milking of the Meibomian glands. However, none of these typical treatments have been clinically effective in treating Blepharitis.

Some non-limiting examples of the disclosure provide advantages to these issues (and others) by providing improved systems and methods for treating eyelid inflammation. For example, some non-limiting examples of the disclosure provide an eyelid treatment system that can include a first substrate (e.g., a lens or contact lens) and one or more light sources optically coupled to the first substrate or coupled to the first substrate. The first substrate can be placed on the eye and the one or more light sources can deliver light (e.g., therapeutic light) to the inner surface of an eyelid to improve inflammation of the eyelid (e.g., to treat posterior blepharitis). The light delivered by the one or more light sources can have one or more wavelengths, with each different wavelength eliciting a different therapeutic response. For example, the light can have a first wavelength that is within a range of 380-470 nanometers (e.g., blue light), which can destroy microbes (e.g., bacteria) of the eyelid, which can cause the blepharitis. As another example, the light can have a second wavelength that is within a range of 620-750 nanometers or 800-1100 nanometers, which can heat the eyelid tissue thereby increasing healing responses (e.g., by increasing blood flow to the region), decreasing pain, etc. As yet another example, the light can have a third wavelength that is within a range of 620-750 nanometers or 800-1100 nanometers, which can decrease inflammation or calm the immune system of the eyelid tissue through photobiomodulation (e.g., decreasing eyelid inflammation by calming the immune system of the eyelid tissue), which can decrease the inflammation of the eyelid tissue (e.g., the light reduces the inflammatory response in the tissue). In some cases, the light including multiple wavelengths can be delivered simultaneously or intermittently. In this way, including when the exact cause of the blepharitis is unknown, the light that can treat multiple causes can be delivered to the eyelid, which can address the blepharitis without requiring knowledge of the exact cause of the blepharitis (e.g., bacteria, yeast, or noninfectious inflammation). In some cases, an inner concave surface of the substrate can partially (or entirely) block light from being transmitted through the inner concave surface to the eye, thereby protecting the eye from potentially damaging light from the light source or ambient light.

In some non-limiting examples, the eyelid treatment system can include a second substrate (e.g., a lens or a contact lens). The second substrate can be similar to the first substrate, and the one or more light sources (e.g., a light source of the one or more light sources) can be optically coupled to the second substrate or coupled to the second substrate. In some cases, the second substrate can be placed over the eye (e.g., with the eyelids closed) and the one or more light sources can deliver the light to the outer surface of the eyelid to improve inflammation of the eyelid (e.g., to treat anterior blepharitis). As described above, the light can have one or more wavelengths with each different wavelength eliciting a different therapeutic response. With the first and second substrates, light can be directed at both the inner surface and the exterior surface of the eyelid, which can ensure that all the surfaces of the eyelid are being adequately treated with light. In some configurations, the first and second substrates can be compressed together, which can express the Meibomian glands (e.g., which can become blocked thereby causing Blepharitis). In some configurations, each of the substrates can include a respective stem, which can facilitate placement of the respective substrate.

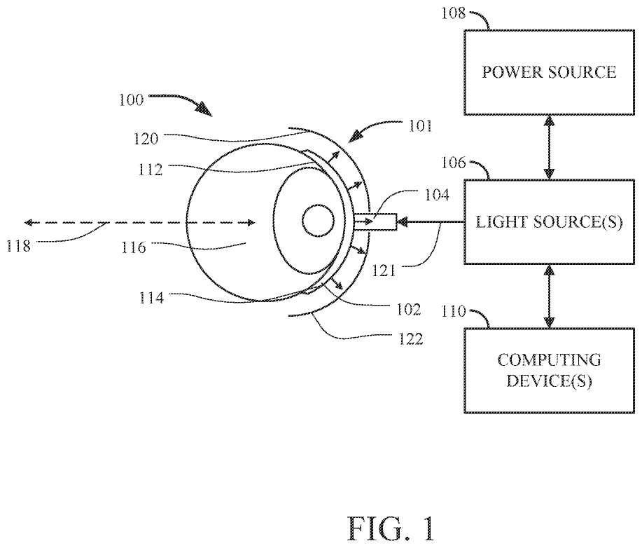

FIG. 1 shows a schematic illustration of a side view of an eyelid treatment system 100. The eyelid treatment system 100 can include a substrate 102, a stem 104 coupled to the substrate 102 that can define an eye device 101, one or more light sources 106, a power source 108, and one or more computing devices 110. The substrate 102 can include an inner concave surface 112 and an outer convex surface 114 opposite the inner concave surface 112. As shown in FIG. 1, the substrate 102 can be placed on an eyeball 116 of the subject (e.g., an eyeball of the subject), similarly to how a contact lens is placed on an eye. The substrate 102 can include a biocompatible material (e.g., the substrate 102 can be formed out of a biocompatible material). In some cases, the biocompatible material can include a hydrogel, a silicone, a polymer, a plastic, an acrylate, etc. In some configurations, the substrate 102 can have a thickness that is less than 1 millimeter (i.e., mm), less than 0.8 mm, less than 0.4 mm, etc., such as at the center of the substrate 102 (e.g., in which the optical axis 118 of the eyeball 116 intersects the center when the substrate 102 is placed on the eyeball 116). In this way, the eye device 101 can be easily placed on the eyeball 116 (e.g., with the substrate 102 mimicking characteristics of a contact lens, which are generally comfortable to wear and many individuals wear contacts and thus have experience placing them on eyeballs). In some configurations, the substrate 102 can have a thickness that is greater than 1 millimeter, greater than 2 millimeters, greater than 3 millimeters, etc., such as at the center of the substrate 102.

Although the substrate 102 is illustrated in FIG. 1 as increasing in thickness along the substrate 102 (e.g., in a direction towards the optical axis 118 of the eyeball 116), such as along the length of the substrate 102, in other configurations, the substrate 102 can have a uniform thickness (and can be curved, so as to create the surfaces 112, 114). In some non-limiting examples, the substrate 102 (and in particular specific portions of the substrate 102) can be transparent, translucent, or can otherwise direct light. Thus, the substrate 102 can be a lens, or more specifically, can be a contact lens. In this way, light from the one or more light sources 106 can be directed towards the substrate 102 and the substrate 102 can disperse the light (e.g., radially), which can be therapeutic light (e.g., eliciting a therapeutic response in the eyelid tissue). For example, the substrate 102 that is a lens can be a diverging lens, such that light emitted from the substrate 102 and towards the inner surface of the eyelid of the subject can reach portions of the eyelid that are further away from the light beam. Although the substrate 102 is illustrated as extending above and below the optical axis 118 of the eyeball 116 (e.g., which can be advantageous in that light can be directed at both eyelids to treat both eyelids), in some configurations, the substrate 102 does not extend below (or above) the optical axis 118. In this way, the substrate 102 could treat the only Blepharitis impacted eyelid, or could treat each eyelid at a time (e.g., by rotating the substrate 102 about the stem 104 to adjust the position of the substrate 102).

In some non-limiting examples, the substrate 102 can block light from reaching the eyeball 116 thereby protecting the cornea, retina, etc., from potentially damaging light. For example, the inner concave surface 112 can at least partially (or entirely) block light from the one or more light sources 106 or the ambient light. For example, as described below, the substrate 102 can include a reflective material, an opaque material, etc., that can block the light at the inner concave surface 112. In some cases, when the substrate 102 includes a reflective material, the reflective material can not only block light from undesirably reaching the eyeball 116 (and other sensitive structures), but the reflective material can also direct light back to the eyelids 120, 122 (e.g., at the inner surfaces of the eyelids 120, 122) to treat the eyelid tissue (e.g., thereby better utilizing the therapeutic light). As another example, the substrate 102 can include a reflective material positioned on the outer convex surface 114 (or a convex surface embedded within the substrate 102). In some cases, the reflective material can be a layer, and a portion of the reflective material can extend above the optical axis 118 of the substrate 102 and can extend below the optical axis 118 of the substrate 102. In this way, the reflective material at a convex surface of the substrate 102 can not only block light (e.g., therapeutic light) from undesirably reaching the sensitive structures of the eyeball 116, but can also redirect the light back at either or both of the eyelids 120, 122.

Although the substrate 102 is illustrated in FIG. 1 as having the shape of a circle, the substrate 102 can have other shapes (e.g., an oval, an ellipse, a disk, other shapes without sharp corners, etc.). In some cases, the substrate 102 can have a single curvature (e.g., a single radius of curvature) or one or more curvatures (e.g., one or more radii of curvature). In some cases, the substrate 102 can be curved in multiple dimensions. In some non-limiting examples, the substrate 102 (e.g., a portion of the substrate 102, such as the non-transparent portion or non-translucent portion) can fluoresce or can be photoluminescent. In other words, the substrate 102 can include a fluorescent or photoluminescent material (e.g., a porphyrin, a metalloporphyrin, etc.), which can be suspended in the larger molecular framework of the substrate 102 (e.g., the fluorescent material can be suspended in a polymer, such as, a hydrogel). The fluorescent or photoluminescent material can absorb first light (e.g., from the one or more light sources 106) of a first frequency (and a first wavelength) and can emit second light of a second frequency smaller than the first frequency (and a second wavelength greater than the first wavelength) towards an inner surface of eyelids 120, 122 of the eyeball 116, the free ends or edges (e.g., inner) of the eyelids 120, 122, the (inner) margins of the eyelids 120, 122, etc. In some cases, the substrate 102 having the fluorescent or photoluminescent material can be advantageous in that, since the fluorescent or photoluminescent material is distributed throughout the substrate 102, each of which essentially defines a light source (i.e., that emits second light) the one or more light sources 106 do not have to deliver light as precisely to the substrate 102 and the substrate 102 does not have to distribute light as effectively (e.g., the one or more light sources 106 could even deliver the first light through the eyelid 120, 122). In some configurations, the photoluminescent material or fluorescent material of the substrate 102 can emit light having the same characteristics (e.g., wavelength) as the light 121 described in detail below that elicits one or more therapeutic effects in the eyelid tissue. In this case, the light 121 having one or more different wavelengths causes the photoluminescent material or fluorescent material of the substrate 102 to emit light having one or more different wavelengths greater than the corresponding wavelength of the light component of the light 121.

In some non-limiting examples, the substrate 102 can be loaded with a therapeutic agent (e.g., a compound, a topical compound, etc.) that can reduce eyelid inflammation when brought into contact with the eyelid. For example, the therapeutic agent can be a steroid, an anti-inflammatory compound (e.g., a calcineurin inhibitor, such as cyclosporine, pimecrolimus, tacrolimus, etc.) an antibiotic, an antiviral, a retinoid (e.g., a topical retinoid that is not a systemic retinoid, including those consumed orally, which can have negative side effects including eye irritation and dry eye), a Janus kinase (“JAK”) inhibitor, etc. For example, when the therapeutic agent is a steroid, the steroid can calm the immune response of the eyelid tissue, which can reduce the eyelid inflammation. As another example, when the therapeutic agent is an antibiotic, the antibiotic can destroy or kill bacteria causing the eyelid inflammation thereby reducing the eyelid inflammation. Similarly, when the therapeutic agent is an antiviral, the antiviral can destroy viruses causing the eyelid inflammation thereby reducing the eyelid inflammation. As yet another example, when the therapeutic agent is a retinoid (or other therapeutic agent that decreases the size of sebaceous glands, dries out sebaceous glands, decreases the production of sebum in the sebaceous glands, etc.) can decrease the size and can limit the production of sebaceous glands (e.g., Meibomian glands) which can help unclog the Meibomian glands that can cause the eyelid inflammation thereby reducing the eyelid inflammation. In some cases, when the substrate 102 is placed on the eyeball 116, the therapeutic agent loaded within the substrate 102 can migrate (e.g., diffuse) to either or both eyelids 120, 122 (e.g., at the inner surface of the respective eyelids 120, 122) to cause the therapeutic effect in either or both eyelids 120, 122 when the therapeutic agent contacts either or both eyelids 120, 122. In some cases, the substrate 102 can block the therapeutic agent from migrating to the eyeball 116. For example, a layer (e.g., a reflective material) that can define the inner concave surface 112 can be substantially impermeable to block migration of the therapeutic agent from migrating into the eyeball 116. In this way, with the substrate 102 loaded with a therapeutic agent, the subject advantageously does not have to take a systemic dosage (e.g., an oral therapeutic agent) of the therapeutic agent. In some cases, the outer convex surface 114 of the substrate 102 can be loaded with the therapeutic agent, which can facilitate faster migration to the eyelids 120, 122, and can help prevent migration into the eyeball 116. In some configurations, the therapeutic agent alone can cause the therapeutic effect to reduce eyelid inflammation or can work in combination (e.g., synergistically) with the therapeutic light to reduce eyelid inflammation by different mechanisms of action. Although the substrate 102 has been described as having a therapeutic agent, the substrate 102 can have a plurality of different therapeutic agents (e.g., loaded on or within the substrate 102), each of which can elicit a different therapeutic effect in the eyelid tissue.

As shown in FIG. 1, the eyelid treatment system 100 can include a stem 104 coupled to the substrate 102, which together can define an eye device 101. The stem 104 can extend away from the substrate 102 in a direction away from the eyeball 116 (e.g., when the substrate 102 is placed on the eyeball 116). In other words, the stem 104 can extend away from the surfaces 112, 114 in a direction from the surface 112 to the surface 114. In some cases, and as illustrated in FIG. 1, the stem 104 can be longer than a thickness of the substrate 102. Although the stem 104 is illustrated as being a cylinder, in other configurations, the stem 104 can have other shapes (e.g., a prism, such as an octagonal prism to more easily grasp the stem 104). The stem 104 can be positioned between the eyelids 120, 122 (e.g., between opposing ends of the eyelids 120, 122, such as when the eyelids 120, 122 are closed). For example, the optical axis 118 of the eyeball 116 can intersect with (and can be substantially, e.g., deviating by less than 10 percent from, being parallel with) a longitudinal axis of the stem 104. In this way, the stem 104 can be easily manipulated by the user or practitioner, such that the eye device 101 can be rotated (e.g., about a longitudinal axis of the stem 104), placed on the eyeball 116, or removed from the eyeball 116.

In some non-limiting examples, the stem 104 can be transparent or translucent and can be optically coupled to the substrate 102 (e.g., at the outer convex surface 114). For example, the stem 104 can include glass (e.g., the stem 104 can be an optical fiber, a waveguide, etc., for light propagation). In this way, the light 121 from the one or more light sources 106 can be emitted into the stem 104, can propagate along the stem 104 to the substrate 102 where the light 121 is directed by the substrate 102 and emitted from the substrate 102 to each eyelid 120, 122. In some cases, the central location of the stem 104 can be advantageous in that the light 121 delivered to the substrate 102 and subsequently directed by the substrate 102 and emitted therefrom (e.g., from a reflective surface defining the inner concave surface 112) can target either or both eyelid margins (e.g., the end of the eyelids) simultaneously if applicable, which can be the source of eyelid inflammation or Blepharitis.

The one or more light sources 106 can be implemented in different ways. For example, each light source 106 can be configured as typically constructed light sources, such as, for example, a laser, a light emitting diode (“LED”), a tungsten-halogen lamp, a mercury or xenon arc lamp, etc. However, some specific implementations of light sources 106 can be more desirable than others, depending on the specific implementation. For example, when the one or more light sources 106 are coupled to the substrate 102 (e.g., one side of the substrate 102), the light sources 106 being implemented as LEDs (e.g., organic LEDs) can be advantageous due to the smaller size, biocompatibility, etc. Each light source 106 can be implemented to provide light having a wavelength of light that corresponds to a desired therapeutic effect in the tissue (e.g., eyelid tissue). For example, the one or more light sources 106 can emit light 121, which is delivered to the substrate 102 (e.g., via the stem 104), and the substrate 102 can direct the light 121 to the eyelids 120, 122 (at the inner surface of the respective eyelid), in which the light 121 causes the therapeutic effect in the eyelid tissue. As another example, the one or more light sources 106 can emit light 121, which is delivered to the substrate 102, and the substrate 102 can emit second light, via photoluminescence of a photoluminescent material of the substrate 102 (or via fluorescence of a fluorescent material of the substrate 102), towards the eyelids 120, 122, in which the second light causes the therapeutic effect in the eyelid tissue.

In some embodiments, therapeutic light delivered to either or both eyelids 120, 122 (e.g., an inner margin thereof at a free end of the respective eyelid) can be a fractional illumination pattern, which can create a distribution of thermally damaged zones of tissue, which can be ablative or non-ablative. In this case, for example, the one or more light sources 106 can be a fractional laser that can create the fractional illumination pattern. In some cases, the substrate 102 and more specifically the outer convex surface 114 of the substrate 102 can direct the fractional illumination pattern at the inner surface of the eyelid. In some cases, the fractional illumination pattern can be directed at a margin (e.g., a free end) of each eyelid 120, 122. In some cases, the fractional illumination pattern can shrink the Meibomian glands of the eyelids 120, 122, or can decrease production of sebum of the Meibomian glands of the eyelids 120, 122. In this way, the fractional illumination pattern can treat Blepharitis caused by over productive, blocked, etc., Meibomian glands.

In other configurations, the therapeutic light delivered to either or both of the eyelids 120, 122 can be selectively absorbed by sebum. For example, the therapeutic light can include a wavelength of light that is the peak absorbance wavelength of sebum (e.g., for wavelengths between substantially 100 nm and 2000 nm). In some cases, the therapeutic light can include a wavelength that is substantially 1726 nm (e.g., a wavelength selective for sebum). In this way, the therapeutic light can vaporize the sebum thereby unblocking the Meibomian glands (e.g., to alleviate Blepharitis), can destroy Meibomian glands, or can shrink the Meibomian glands.

In some non-limiting examples, the therapeutic effect can be reducing inflammation of an eyelid (e.g., improving Blepharitis of the eyelid), reducing inflammation of an eye (e.g., from reducing inflammation of the eyelid), destroying microbes (e.g., pathogens) of the eyelid (e.g., destroying bacteria), improving a healing response of the eyelid (e.g., by heating the eyelid), heating the eyelid (e.g., thereby improving an immune response of the eyelid tissue), improving the blood flow of the eyelid tissue (e.g., by heating the tissue, such as from the light 121), decreasing the pain of the eyelid tissue (e.g., by heating the tissue, such as from the light 121), improving an immune response of the eyelid tissue (e.g., by heating the tissue, such as from the light 121), causing photobiomodulation of the eyelid tissue, etc. Thus, in some configurations, the light 121 can include multiple wavelengths of light, each of which when delivered to the eyelid tissue elicits a different therapeutic effect in the eyelid tissue. In some configurations, the light 121 can include light having a wavelength of light that elicits multiple therapeutic responses in the eyelid tissue (e.g., near infrared light can decrease inflammation or calm the immune system of the eyelid tissue through photobiomodulation and can heat the eyelid tissue).

In some non-limiting examples, each light source 106 can emit light having a different wavelength each eliciting a different therapeutic response in the eyelid tissue. In some configurations, the light source 106 can emit light having multiple different wavelengths. For example, the light source 106 can emit the light 121 that includes blue light, red light, infrared light (e.g., near infrared light), etc. In some cases, the light 121 can include other wavelengths (or other light components) that are extraneous that do not necessarily elicit a therapeutic effect in the eyelid tissue (e.g., when the light 121 is white light). In other cases, the light 121 only includes wavelengths that each elicit a therapeutic effect in the eyelid tissue. For example, the light source 106 can include a plurality of optical paths, and each optical path can include an optical component positioned within the optical path (e.g., that can alter the wavelength of the light that passes through the optical component), such as an optical filter (e.g., a near infrared filter, a red light filter, a blue light filter, etc.). In this way, one light source can be used to deliver multiple different wavelengths, and the wavelengths can be within a tight wavelength range dictated by the optical filter. As another example, a first light source 106 can deliver first light (e.g., of the light 121) having a first wavelength of light that elicits a first therapeutic effect in the eyelid tissue, a second light source 106 can deliver second light having a second wavelength of light that elicits a second therapeutic effect (different from the first therapeutic effect) in the eyelid tissue, and so on. In this configuration, with each light source tailored to a specific wavelength of light (or a wavelength range or band of light), other light parameters may be more easily adjusted (e.g., the light flux or intensity of the light emitted by the light source). In some configurations, the light delivered to an eyelid (e.g., the light 121) can have an irradiance of substantially 300 mW/cm², which can be substantially twice the irradiance of sunlight.

In some non-limiting examples, the light 121 (or the light delivered to the eyelid tissue, such as when the substrate 102 includes a photoluminescent material) can include violet light having a wavelength within a wavelength range of substantially (e.g., deviating by less than 10 percent from) 380 nanometers (i.e., nm) to substantially 450 nm, blue light having a wavelength within a wavelength range of substantially 450 nm to substantially 485 nm, red light having a wavelength within a wavelength range of substantially 625 nm to substantially 750 nm, near infrared light having a wavelength within a wavelength range of substantially 700 nm to substantially 1000 nm or substantially 1100 nm. In some cases, the light 121 (or the therapeutic light delivered to the eyelid tissue, such as when the substrate 102 includes a photoluminescent material) can include light (i.e., violet light or blue light) having a wavelength within a wavelength range of substantially 400 nm to 470 nm, red light having a wavelength within a wavelength range of substantially 620 nm to substantially 750 nm, infrared light (e.g., near infrared light) having a wavelength within a wavelength range of substantially 800 nm to substantially 1100 nm. In some cases, the light 121 (or therapeutic light) can have a wavelength of substantially 1064 nm (e.g. corresponding to photobiomodulation). In some configurations, the light 121 (or therapeutic light) can be ultraviolet light (e.g., UVA light, UVB light, etc.). For example, the light 121 (or the therapeutic light) can be within a wavelength range of substantially 100 nm to substantially 400 nm, in a wavelength range of substantially 315 nm to substantially 400 nm (e.g., corresponding to UVA light), in a wavelength range of substantially 280 nm to substantially 315 nm, etc. In some configurations, UV light that is the therapeutic light applied to the eyelid(s) 120, 122 can be advantageous in that the UV light can kill pathogens (e.g., including those that can be causing the Blepharitis), and the UV light can decrease inflammation (e.g., by calming the immune system) for conditions including eczema and psoriasis each of which can cause or worsen Blepharitis. Although the wavelength ranges have been described as being substantially within the number ranges, it is appreciated that the wavelength ranges can be exact ranges (e.g., substantially 380 nm to substantially 450 nm can be 380-450 nm, 342 nm to 450 nm, etc.).

In some non-limiting examples, when the light 121 (or light delivered to the eyelid tissue) includes multiple different wavelengths (with each eliciting a different therapeutic effect), the subcomponents of the light 121 (e.g., that has a different wavelength) can be emitted simultaneously or sequentially (e.g., iteratively). For example, the blue light of the light 121 (or light delivered to the eyelid tissue) can be emitted by the one or more light sources 106 and delivered to the eyelid tissue at a first time, and the near infrared light of the light 121 (or light delivered to the eyelid tissue) can be emitted by the one or more light sources 106 and delivered to the eyelid tissue at a second time, which can overlap with the first time (e.g., corresponding to simultaneously) or can be separate from the first time (e.g., corresponding to sequentially).

In some non-limiting examples, the eyelid treatment system 100 can include the power source 108, which can supply power to some or all of the specific components of the eyelid treatment system 100. For example, the power source 108 can be an electrical power source (e.g., an electrical storage device, a battery, a power cord, etc.) and can provide power to the one or more light source 106, the computing device 110, etc. In some non-limiting examples, the power source 108 can be coupled to the eye device 101 (e.g., when the one or more light sources 106 are coupled to the substrate 102), which can be advantageous in that the spatial footprint of the eye device 101 can be greatly reduced. For example, the power source 108 can be coupled to the stem 104, for example, at an end of the stem 104 positioned away from the substrate 102 (e.g., where the power source 108 is positioned further away from the eyeball 116 than the substrate 102, when the substrate 102 is placed on the eyeball 116). In this way, the power source 108, which may not be biocompatible, does not undesirably interfere with the eyeball 116 or eyelid tissue. In some cases, the power source 108 can be coupled to the substrate 102 (e.g., at the outer convex surface 114). In some cases, including when the power source 108 is not biocompatible, the power source 108 can be encapsulated (e.g., with an epoxy, polymer, hydrogel, etc.) so as to avoid direct contact with tissue and the power source 108. In some configurations, the power source 108 can be an electrical storage device, such as a battery. More specifically, the battery can be a coin cell battery, which can be fairly small (as compared to other batteries).

In some non-limiting examples, the eyelid treatment system 100 can include the one or more computing devices 110, each of which can implement some or all of the processes (or tasks) of the methods described herein, as applicable. In particular, the computing device 110 can cause each of the components of the eyelid treatment system 100 to implement a particular task (e.g., by sending instructions to a respective component). For example, the computing device 110 can cause the one or more light sources 106 to emit the light 121 to treat the eyelid tissue (e.g., the light 121 itself treating the eyelid tissue), or the light 121 causing the photoluminescent material of the substrate 102 to emit light towards the eyelid tissue that treats the eye lid tissue. Although the description of the computing device 110 refers to a computing device, this description is applicable to all of the computing devices of the eyelid treatment system 100 when implemented with more than one computing device. The computing device 110 can include typical computing components, such as, a processor device, memory, communication systems, a display, inputs (e.g., a mouse, a keyboard, a touch screen, sensors, and the like), power sources, and the like. In some cases, the computing device 110 can take on a variety of specific forms including a desktop, a laptop, a mobile device (e.g., a tablet, or a smartphone), and the like. For example, in some cases, the computing device 110 can be positioned external to the eye device 101, or can be coupled to the eye device 101 (e.g., when the power source and one or more light sources are coupled to the substrate 102).

In some non-limiting examples, the parameters of the light 121 (and light components thereof) can be changed, such as, for example, by the computing device 110. For example, the light flux, intensity, duration, etc., of the light components from each of the one or more light sources 106 can be changed (e.g., by the computing device 110), such as depending on the severity of the eyelid inflammation, chronic evidence of eyelid inflammation, etc.

In some non-limiting examples, the computing device 110 can include a processor device, memory, communication systems, and the like, so as to communicate with another computing device (e.g., a desktop computer), not shown in FIG. 1. In other cases, the computing device 110 can simply be implemented as a processor. In some non-limiting examples, another computing device (e.g., a smartphone) of the eyelid treatment system 100 (not shown) can cause the computing device 110 of the eyelid treatment system 100 to implement some or all of the steps of the processes described herein, as applicable.

As shown in FIG. 1, the eye device 101 can be placed on the eyeball 116 of a subject prior to treatment of the eyelids 120, 122 (or single eyelid). As described above, the substrate 102 of the eye device 101 can be a contact lens, or can be structured in a similar way as a contact lens (e.g., being formed out of a polymer, such as a hydrogel, having a thickness similar to a contact lens, etc.). When the substrate 102 is placed on the eyeball 116, the inner concave surface 112 can be in direct contact with the eyeball 116, and the outer convex surface 114 can be positioned away from the eyeball 116 and towards the eyelids 120, 122. Once the substrate 102 is placed on the eyeball 116, the subject can close their eyelids 120, 122 and the treatment can begin. In this case, the one or more light sources 106 can deliver the light 121 to the substrate 102, which directs the light 121 (or emits secondary light, such as when the substrate 102 includes a photoluminescent material) towards the eyelids 120, 122 (e.g., the inner surface of the eyelids 120, 122, the margin of one or more eyelids including the ends thereof) thereby treating the eyelids 120, 122 (e.g., by eliciting a therapeutic effect in the eyelid from the light applied thereto). In some non-limiting examples, the eyelid treatment system 100 can be used to treat posterior Blepharitis of the eyelids 120, 122 since the light directed by the substrate can better target the inner edge of the eyelids 120, 122, which can be the concentrated source of posterior Blepharitis issues.

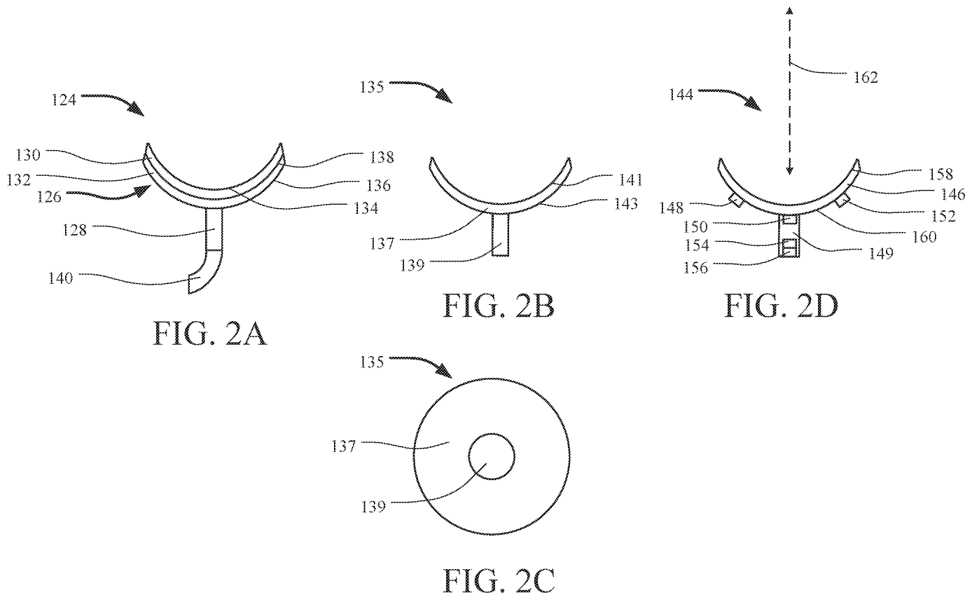

FIG. 2A shows a schematic illustration of a side view of an eye device 124, which can be implemented in a similar manner as the eye device 101. Thus, the description of the eye device 124 pertains to the eye device 101 (and vice versa). For example, the eye device 124 can include a substrate 126 and a stem 128 coupled to the substrate 126. The substrate 126 can include layers 130, 132. The layers 130, 132 can be positioned on top of each other and the layer 130 can define an inner concave surface 134, while the layer 132 can define an outer convex surface 136. The layer 130 can partially or entirely block light from passing therethrough to the eye. For example, the layer 130 can be formed out of an opaque material (e.g., a material with a high light absorption, such as tungsten). In this case, the opaque material can be a dark color (e.g., black) so as to absorb a large portion of the light from a light source or ambient light (e.g., environmental light), which would thus block light from passing through to the eye. In some cases, the layer 130 can be an optical filter that is configured to prevent light of a particular wavelength from passing through to the eyeball. For example, the optical filter can be configured to prevent light having the same wavelength as the therapeutic light from passing therethrough. As another example, the layer 130 can be formed out of a reflective material, which can reflect a portion (or all of the light) directed at the reflective material. In some cases, the reflective material can be a metal (e.g., chromium), a ceramic, etc. Unlike the layer 130, the layer 132 can be translucent or transparent so as to direct light. For example, the layer 130 can define a lens which can diverge light, such as light reflected off the layer 130. In some configurations, and as illustrated, the layer 130 can define an inner convex surface 138 positioned below the layer 132, which can provide a reflective surface for which light can reflect off of the layer 130.

Although the layer 130 is illustrated as extending across the entire surface of the layer 132, in some configurations, the layer 130 can extend partially across the layer 132. Similarly, although the layer 132 is illustrated as extending across the entire surface of the layer 130, in some configurations, the layer 132 can extend partially across the layer 130. In this configuration, the layer 130 can extend beyond an end of the layer 132 at one end of the eye device 124 (e.g., left of the stem 128 in FIG. 2A) and can extend beyond an end of the layer 132 at another end of the eye device 124 (e.g., right of the stem 128 in FIG. 2A). In this way, the layer 130 can ensure that peripheral light is prevented from reaching the eye. In some configurations, each end of the substrate 126 can include a reflective material. For example, a first end of the substrate 126 (e.g., the left side of FIG. 2A) can include a reflective material and a second end of the substrate 126 (e.g., the right side of FIG. 2A) can include a reflective material. In this way, light that would otherwise be emitted from the ends is reflected away from the end, which can avoid light from entering the eyeball, such as when the substrate 126 is relatively small in width.

In some non-limiting examples, the stem 128 can be transparent or translucent and can be optically coupled to the substrate 126 at the layer 132. For example, the stem 128 can include glass (e.g., the stem 128 can be an optical fiber, a waveguide, etc., for light propagation). In some configurations, an optical component 140 can be coupled to the stem 128 (e.g., and in some cases can be removably coupled to the stem 128). The optical component 140 can guide light (from the light source(s)) to the stem 128 and then to the substrate 126. The optical component 140 can be implemented in different ways. For example, as illustrated, the optical component 140 can be an optical fiber. In other configurations, the optical component 140 can be a waveguide, an optical guide, etc. In some non-limiting examples, the optical component 140 being coupled to the stem 128 can be advantageous in that the eye device 124 can be structured in a relatively simple manner (e.g., does not require light sources, power sources, computing devices, etc., coupled to the substrate 126 or the stem 128). In addition, in some cases, the optical component 140 being removably coupled to the stem 128 can be advantageous in that the eye device 124 can be removed, cleaned (e.g., by placing in an autoclave), and reused again without forcing components (such as light sources) that may be unable to be adequately cleaned into contact with bodily fluids. Similarly, the optical component 140 being removably coupled to one or more light sources, such as the one or more light sources 106 can be advantageous in that, again, the one or more light sources can simply be disconnected from the optical fiber and used subsequently for a different procedure without the light sources being in direct contact with bodily fluids.

FIG. 2B shows a schematic illustration of a side view of an eye device 135, which can be implemented in a similar manner as the other eye devices disclosed herein (e.g., the eye device 101). Thus, the description of the eye device 135 pertains to the other eye devices described herein (and vice versa). The eye device 135 can include a substrate 137 and a stem 139 coupled to the substrate 137. The substrate 137 can define an inner concave surface 141 and an outer convex surface 143 opposite the inner concave surface 141. In some configurations, the substrate 137 itself can partially or entirely block light from passing therethrough to the eye. For example, the substrate 137 can have optical properties, such that light is blocked from passing through the inner concave surface 141 to the eye. As a more specific example, the substrate 137 can be formed out of a material (e.g., a polymer) and can have a thickness, such that light is totally internally reflected and thus light propagates along the thickness of the substrate 137. In some cases, one or more imperfections (e.g., divots, scratches, etc.) can be included on or into the substrate 137 (e.g., except for the inner concave surface 141), such that when light that is totally internally reflected and reaches an imperfection, the light stops being totally internally reflected and radiates away (in all directions) away from the imperfection. In this way, light can be emitted out from the outer convex surface 143 and delivered towards an eyelid, thereby treating the eyelid tissue. In this example, the light entering the substrate 137 would enter at an angle that allows for total internal reflection of the light. For example, the stem 139 can be angled relative to the substrate 137, such that light entering and directed by the stem 139 along with stem enters the substrate 137 at an angle that allows for total internal reflection of the light along the substrate 137.

FIG. 2C shows a schematic illustration of a top view of the eye device 135. As shown in FIG. 2C, the substrate 137 of the eye device 135 is circular (e.g., has a circular shape when viewed from the top view, as in FIG. 2C). However, in other configurations, the substrate 137 can have other shapes. Similarly, although the stem 139 of the eye device 135 is illustrated as being cylindrical in FIG. 2C, the stem 139 can have other shapes.

FIG. 2D shows a schematic illustration of a side view of an eye device 144, which can be implemented in a similar manner as the other eye devices disclosed herein (e.g., the eye device 101). Thus, the description of the eye device 144 pertains to the other eye devices described herein (and vice versa). The eye device 144 can include a substrate 146 defining an inner concave surface 158 and an outer convex surface 160 opposite the inner concave surface 158, a stem 149 coupled to the substrate 146, light sources 148, 150, 152, a power source 154, and a computing device 156. As shown in FIG. 2D, each light source 148, 150, 152 can be coupled to the substrate 146 at an outer convex surface 160 of the substrate 146 and can emit light in a direction away from the eye (and towards an eyelid of the eye), or in other words, in a direction from the inner concave surface 158 and to the Outer convex surface 160. Each light source 148, 150, 152 can be implemented in a similar manner as the light sources described herein (e.g., the one or more light sources 106) and can be distributed along the substrate 146 in different ways. For example, the light source 148 can be positioned on one side of the substrate 146, the light source 152 can be positioned on an opposing side of the substrate 146, and the light source 150 can be positioned between the light sources 148, 152. In some cases, the light source 150 can be aligned with the center of the substrate 146, which can define an optical axis 162 of the substrate 146 (e.g., when the substrate 146 is implemented as a lens, such as a contact lens), such that the optical axis 162 intersects with the center of the substrate 146. Similarly, the light source 150 can be positioned such that the light source 150 is aligned with the stem 149, or the light source 150 can be substantially parallel to a longitudinal axis of the stem 149. In some cases, the light source 150 can be coupled to the stem 149 (e.g., at a side of the stem 149). In other cases, the light source 150 can be coupled to the stem 149 at a free end of the stem 149 (e.g., opposite the end of the stem 149 coupled to the substrate 146). In this way, the light source 150 (including when the eye device 144 does not include the other light sources 148, 152), can deliver therapeutic light through the stem 149 and to the substrate 146 where the substrate 146 disperses the therapeutic light to the eyelid, such that the light sources do not impede dissemination of the therapeutic light. Regardless of the configuration, the light source 150 positioned at the center of the substrate 146 can be advantageous in that light from the light source 150 can be highly targeted to the eyelid margins (e.g., the end of the eyelids), which can be the source of eyelid inflammation or Blepharitis. Also, the light from the light source 150 can treat both eyelid margins simultaneously.

Although the eye device 144 is illustrated as having three light sources 148, 150, 152, in other configurations, the eye device 144 can include other numbers of light sources (e.g., two, four, five, etc.). FIG. 2D shows the light sources 148, 150, 152 coupled to the substrate 146 at the outer convex surface 160, however, each light source 148, 150, 152 can be coupled to the substrate 146 in a different manner. For example, each light source 148, 150, 152 can be embedded within the substrate 146, coupled to the inner concave surface 158 of the substrate 146, etc. In this case, each light source 148, 150, 152 can be positioned within or aligned with a respective aperture or recess in the substrate 146. In addition, each aperture (or recess) can be covered by a transparent or translucent material, so as to allow light through to the eyelid, but also blocking debris (e.g., dust, dirt, etc.) from negatively impacting the respective light source. In some cases, the light source positioned within a recess of a substrate can be advantageous in that the light source does not directly contact the eye, which could irritate the eye.

As shown in FIG. 2D, the eye device 144 can include the power source 154 and the computing device 156, each of which can be implemented in a similar manner as the other power sources and computing devices described herein. In some cases, the power source 154 can be coupled to the stem 149 and similarly, the computing device 156 can be coupled to the stem 149. In particular, the power source 154 and the computing device 156 can be coupled to the side of the stem 149, or can be coupled to an end of the stem 149 (e.g., opposite the end of the stem 149 coupled to the substrate 146). In other cases, the power source 154 and the computing device 156 can be embedded within the stem 149 (e.g., positioned within a recess of the stem 149). Regardless of the configuration, the power source 154 (and the computing device 156) coupled to the stem 149 can be advantageous in that these components are positioned so that they do not block light from reaching an eyelid (e.g., as opposed to being coupled to the substrate 146) and the stem 149 is thicker and larger than the substrate 146, such that the stem 149 provides a larger spatial footprint for these components. In some cases, the light source 148, 150, 152 can be electrically connected to the power source 154 and the computing device 156 (e.g., via one or more wires, which can be embedded within the substrate 146), and the power source 154 can be electrically connected to the computing device 156 (e.g., via one or more wires, which can be embedded within the stem 149). In some cases, the central positioning of the power source 154 and the computing device 156 can be advantageous in that due to the central location relative to the light sources 148, 150, 152, the length of the wires are advantageously shorter than other positional relationships.

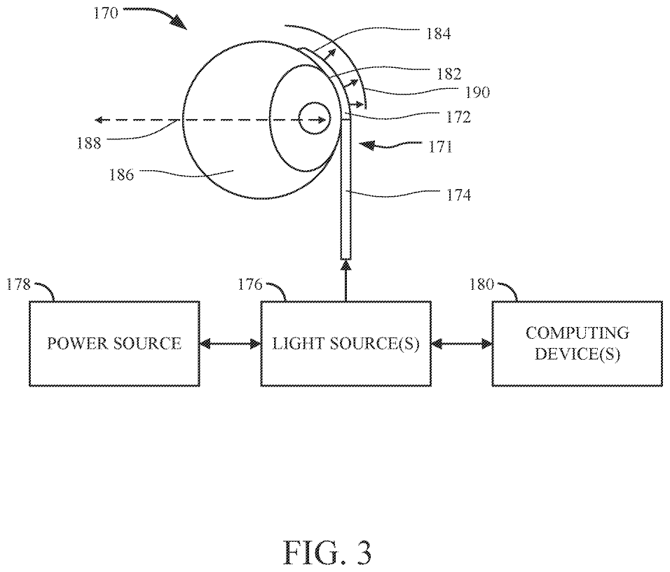

FIG. 3 shows a schematic illustration of a side view of an eyelid treatment system 170, which can be implemented in a similar manner as the other eyelid treatment systems described herein (e.g., the eyelid treatment system 100). Thus, the description of the eyelid treatment system 170 pertains to the other eyelid treatment systems described herein (and vice versa). The eyelid treatment system 170 can include a substrate 172, a stem 174 coupled to the substrate 172 that can define an eye device 171, one or more light sources 176, a power source 178, and one or more computing devices 180. The substrate 172 can include an inner concave surface 182 and an outer convex surface 184 opposite the inner concave surface 182. As shown in FIG. 3, the substrate 172 can be placed on an eyeball 186 of the subject (e.g., an eyeball of the subject), similarly to how a contact lens is placed on an eye. As shown in FIG. 3, the stem 174 can be coupled to the substrate 172, where an end of the stem 174 is coupled to an end of the substrate 172. In some cases, the stem 174 is longer than the substrate 172, which can facilitate easy placement of the substrate 172 into contact with the eyeball 186. In some configurations, the stem 174 extends away from the substrate 172 in a direction away from the end of the substrate 172 (e.g., that is coupled to the stem 174). In other words, a longitudinal axis of the stem 174 can be substantially parallel to an optical axis 188 of the eyeball 186 when the substrate 172 is placed on the eyeball 186. In this way, the stem 174 can be grasped by a practitioner and easily slid under the eyelid 190 of the subject. Similarly to the stem 104 described previously, the stem 174 can be transparent or translucent and can be optically coupled to the substrate 102 and can propagate light from the one or more light sources 176 to the substrate 102.

As shown in FIG. 3, the substrate 172 curves and extends above the optical axis 188 of the eyeball 186, but does not curve to extend below the optical axis 188 of the eyeball 186. In this way, the eyelid treatment system 170 can treat only the eyelid 190 (i.e., the upper eyelid) and not the lower eyelid, when, for example, only the eyelid 190 is suffering from Blepharitis or eyelid inflammation. In some configurations, however, the substrate 172 can be configured, structured, etc., to treat only the other eyelid (and not the eyelid 190). In this case, the substrate 172 can curve to extend below the optical axis 188 of the eyeball 186, but does not curve to extend above the optical axis 188 of the eyeball 186. In this way, the eyelid treatment system 170 can treat only the other eyelid (i.e., the lower eyelid) and not the upper eyelid (i.e., the eyelid 190), when, for example, only the lower eyelid is suffering from Blepharitis or eyelid inflammation. In this case, the stem 174 can be coupled to the substrate 172 at a curve of the substrate 172 (e.g., the center of the substrate 172).

As shown in FIG. 3, the substrate 172 can be placed on the eyeball 186 (e.g., the eyeball), by, for example, grasping the stem 174. Then, light from the one or more light sources 176 can be directed into the stem 174, can propagate along the stem 174 and enter the substrate 172, where the substrate 172 emits the light out therefrom to the eyelid 190 (e.g., the inner surface of the eyelid 190, the margin of the eyelid, for example, at the inner surface of the eyelid 190, etc.) to treat the eyelid 190. Although the eyelid treatment system 170 is illustrated as having the one or more light sources 176 direct light into the stem 174, in other configurations, the one or more light sources 176 can be coupled to the substrate 172 (e.g., an outer convex surface of the substrate 172).

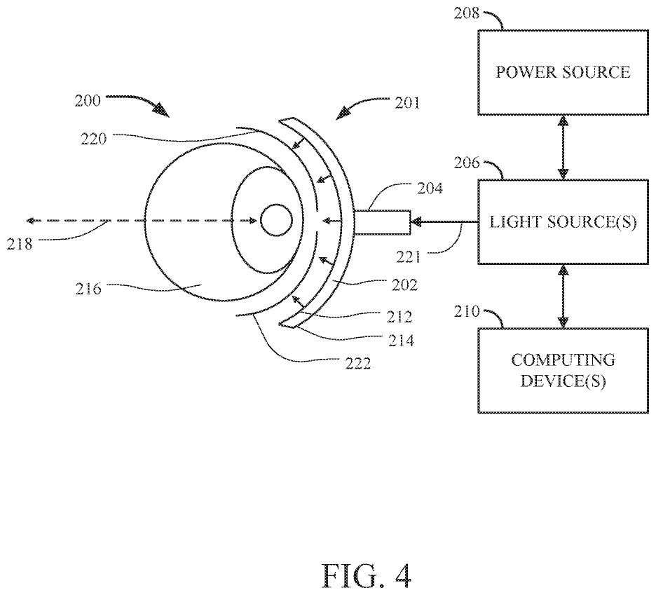

FIG. 4 shows a schematic illustration of a side view of an eyelid treatment system 200, which can be implemented in a similar manner as the other eyelid treatment systems described herein (e.g., the eyelid treatment system 100). Thus, the description of the eyelid treatment system 200 pertains to the other eyelid treatment systems described herein (and vice versa). The eyelid treatment system 200 can include a substrate 202, a stem 204 coupled to the substrate 202 that can define an eye device 201, one or more light sources 206, a power source 208, and one or more computing devices 210. The substrate 102 can include an inner concave surface 212 and an outer convex surface 214 opposite the inner concave surface 212. As shown in FIG. 4, the substrate 202 can be placed over an eyeball 216 of the subject and over the eyelids 220, 222 of the eyeball 216. In some cases, the substrate 202 can be thicker than the substrate 102 of the eyelid treatment system 100 since the substrate 202 can be external to the eyeball 216 (e.g., the eyeball) to treat the eyelids 220, 222. Thus, the substrate 202 does not need to be made thin so as to make the substrate 202 comfortable for direct contact with the eyeball 216. Similarly, the substrate 202 can be formed out of a different material than the substrate 102, again, because the substrate 202 does not have to be in direct contact with the eyeball 216. Thus, the substrate 202 can be formed out of a non-biocompatible material.

Although the substrate 202 is illustrated in FIG. 1 as increasing in thickness along the substrate 202 (e.g., in a direction towards the optical axis 218 of the eyeball 216), such as along the length of the substrate 202, in other configurations, the substrate 202 can have a uniform thickness (and can be curved, so as to create the surfaces 212, 214). In some non-limiting examples, the substrate 202 (and in particular specific portions of the substrate 202) can be transparent, translucent, or can otherwise direct light. Thus, the substrate 202 can be a lens. In this way, light from the one or more light sources 206, ), which can be therapeutic light (e.g., eliciting a therapeutic response in the eyelid tissue), can be directed towards the substrate 202 and the substrate 202 can disperse the light (e.g., radially). For example, the substrate 202 that is a lens can be a converging lens, such that light emitted from the substrate 202 and towards the inner surface of the eyelid of the subject can ensure that light is directed at the eyelids 220, 222, rather than being dispersed away from the eyelids 220, 222. Although the substrate 202 is illustrated as extending above and below the optical axis 218 of the eyeball 216 (e.g., which can be advantageous in that light can be directed at both eyelids to treat both eyelids), in some configurations, the substrate 202 does not extend below (or above) the optical axis 218. In this way, the substrate 202 could treat the only Blepharitis impacted eyelid, or could treat each eyelid at a time (e.g., by rotating the substrate 202 about the stem 204 to adjust the position of the substrate 202).

In some non-limiting examples, the substrate 202 can block ambient light (e.g., light from the environment surrounding the eye device 201) from reaching the eyeball 116 thereby protecting the cornea, retina, etc., from potentially damaging light. For example, the outer convex surface 214 can at least partially (or entirely) block ambient light from being transmitted therethrough. As a more specific example, the substrate 202 can include a reflective material, an opaque material, etc., that can block the light at the outer convex surface 214. In some cases, when the substrate 202 includes a reflective material, the reflective material can not only block ambient light from undesirably reaching the eyeball 216 (and other sensitive structures), but the reflective material can also direct the light from the one or more light sources 206 back to the eyelids 220, 222 (e.g., at the outer surfaces of the eyelids 220, 222) to treat the eyelid tissue (e.g., thereby better utilizing the therapeutic light). As another example, the substrate 202 can include a reflective material positioned on the inner concave surface 212 (or a concave surface embedded within the substrate 202). In some cases, the reflective material can be a layer, and a portion of the reflective material can extend above the optical axis 118 of the substrate 102 and can extend below the optical axis 118 of the substrate 102. In this way, the reflective material at a concave surface of the substrate 102 can not only block light (e.g., therapeutic light) from undesirably reaching the sensitive structures of the eyeball 116, but can also redirect the light back at either or both of the eyelids 220, 212.

Although the substrate 202 is illustrated in FIG. 1 as having the shape of a circle, the substrate 202 can have other shapes (e.g., an oval, an ellipse, a disk, other shapes without sharp corners, etc.). In some cases, the substrate 202 can have a single curvature (e.g., a single radius of curvature) or one or more curvatures (e.g., one or more radii of curvature). In some cases, the substrate 202 can be curved in multiple dimensions. In some configurations, the substrate 202 can have a curvature that is larger than a curvature of the substrate 102, since the substrate 202 is positioned exterior to the eye as opposed to the substrate 102. In some non-limiting examples, the substrate 202 (e.g., a portion of the substrate 202, such as the non-transparent portion or non-translucent portion) can fluoresce or can be photoluminescent. In other words, the substrate 202 can include a fluorescent or photoluminescent material (e.g., a porphyrin, a metalloporphyrin, etc.), which can be suspended in the larger molecular framework of the substrate 202 (e.g., the fluorescent material can be suspended in a polymer, such as a hydrogel). The fluorescent or photoluminescent material can absorb first light (e.g., from the one or more light sources 206) of a first frequency (and a first wavelength) and can emit second light of a second frequency smaller than the first frequency (and a second wavelength greater than the first wavelength) towards an outer surface of the eyelids 120, 122 of the eyeball 116, the free ends or edges (e.g., outer) of the eyelids 120, 122, the (outer) margins of the eyelids 120, 122, etc. In some cases, the substrate 202 having the fluorescent or photoluminescent material can be advantageous in that, since the fluorescent or photoluminescent material is distributed throughout the substrate 202, each of which essentially defines a light source (i.e., that emits second light) the one or more light sources 206 do not have to deliver light as precisely to the substrate 202 and the substrate 202 does not have to distribute light as effectively. In some configurations, the photoluminescent material or fluorescent material of the substrate 202 can emit light having the same characteristics (e.g., wavelength) as the light 221 described in detail below that elicits one or more therapeutic effects in the eyelid tissue. In this case, the light 221 having one or more different wavelengths causes the photoluminescent material or fluorescent material of the substrate 202 to emit light having one or more different wavelengths greater than the corresponding wavelength of the light component of the light 221.

In some non-limiting examples, the substrate 202 can be loaded with a therapeutic agent (e.g., a compound, a topical compound, etc.) that can reduce eyelid inflammation when brought into contact with the eyelid. For example, the therapeutic agent can be a steroid, an anti-inflammatory compound (e.g., a calcineurin inhibitor, such as cyclosporine, pimecrolimus, tacrolimus, etc.) an antibiotic, an antiviral, a retinoid, (e.g., a topical retinoid that is not a systemic retinoid, including those consumed orally, which can have negative side effects including eye irritation and dry eye), a Janus kinase (“JAK”) inhibitor, etc. For example, when the therapeutic agent is a steroid, the steroid can calm the immune response of the eyelid tissue, which can reduce the eyelid inflammation. As another example, when the therapeutic agent is an antibiotic, the antibiotic can destroy or kill bacteria causing the eyelid inflammation thereby reducing the eyelid inflammation. Similarly, when the therapeutic agent is an antiviral, the antiviral can destroy viruses causing the eyelid inflammation thereby reducing the eyelid inflammation. As yet another example, when the therapeutic agent is a retinoid (or other therapeutic agent that decreases the size of sebaceous glands, dries out sebaceous glands, decreases the production of sebum in the sebaceous glands, etc.) can decrease the size and can limit the production of sebaceous glands (e.g., Meibomian glands) which can help unclog the Meibomian glands that can cause the eyelid inflammation thereby reducing the eyelid inflammation. In some cases, when the substrate 102 is placed over either or both of the eyelids 220, 222 (and over the eyeball 116), the therapeutic agent loaded within the substrate 102 can migrate (e.g., diffuse) to either or both eyelids 120, 122 (e.g., at the outer surface of the respective eyelids 220, 222) to cause the therapeutic effect in either or both eyelids 220, 222 when the therapeutic agent contacts either or both eyelids 220, 222. In some configurations, with the substrate 102 loaded with a therapeutic agent, the subject advantageously does not have to take a systemic dosage (e.g., an oral therapeutic agent) of the therapeutic agent. In some cases, the inner concave surface 212 of the substrate 202 can be loaded with the therapeutic agent, which can facilitate faster migration to the eyelids 220, 222. In some configurations, the therapeutic agent alone can cause the therapeutic effect to reduce eyelid inflammation or can work in combination (e.g., synergistically) with the therapeutic light to reduce eyelid inflammation by different mechanisms of action. Although the substrate 202 has been described as having a therapeutic agent, the substrate 202 can have a plurality of different therapeutic agents (e.g., loaded on or within the substrate 202), each of which can elicit a different therapeutic effect in the eyelid tissue.

As shown in FIG. 4, the eyelid treatment system 200 can include a stem 204 coupled to the substrate 202, which together can define the eye device 201. The stem 204 can extend away from the substrate 202 in a direction away from the eyeball 216 (e.g., when the substrate 202 is placed on the eyeball 216). In other words, the stem 204 can extend away from the surfaces 212, 214 in a direction from the surface 212 to the surface 214. In some cases, and as illustrated in FIG. 4, the stem 204 can be longer than a thickness of the substrate 202. Although the stem 204 is illustrated as being a cylinder, in other configurations, the stem 204 can have other shapes (e.g., a prism, such as an octagonal prism to more easily grasp the stem 204). The stem 204 can be positioned between the eyelids 220, 222 (e.g., between opposing ends of the eyelids 220, 222, such as when the eyelids 220, 222 are closed). For example, the optical axis 218 of the eyeball 216 can intersect with (and can be substantially, e.g., deviating by less than 10 percent from, being parallel with) a longitudinal axis of the stem 204. In this way, the stem 204 can be easily manipulated by the user or practitioner, such that the eye device 201 can be rotated (e.g., about a longitudinal axis of the stem 204), placed on the eyeball 216, or removed from the eyeball 216.

In some non-limiting examples, the stem 204 can be transparent or translucent and can be optically coupled to the substrate 202 (e.g., at the outer convex surface 214). For example, the stem 204 can include glass (e.g., the stem 204 can be an optical fiber, a waveguide, etc., for light propagation). In this way, the light 221 from the one or more light sources 206 can be emitted into the stem 204, can propagate along the stem 204 to the substrate 202 where the light 221 is directed by the substrate 202 and emitted from the substrate 202 to each eyelid 220, 222. In some cases, the central location of the stem 204 can be advantageous in that the light 121 delivered to the substrate 102 and subsequently directed by the substrate 102 (e.g., passing through the substrate 202) can target either or both eyelid margins (e.g., the ends of the eyelid) simultaneously if applicable, which can be the source of eyelid inflammation or Blepharitis.

The one or more light sources 206 can be implemented in different ways. For example, each light source 206 can be configured as typically constructed light sources, such as, for example, a laser, a light emitting diode (“LED”), a tungsten-halogen lamp, a mercury or xenon arc lamp, etc. However, some specific implementations of light sources 206 can be more desirable than others, depending on the specific implementation. For example, when the one or more light sources 206 are coupled to the substrate 202 (e.g., one side of the substrate 102), the light sources 206 being implemented as LEDs (e.g., organic LEDs) can be advantageous due to the smaller size, biocompatibility, etc. Each light source 206 can be implemented to provide light having a wavelength that corresponds to a desired therapeutic effect in the tissue (e.g., eyelid tissue). For example, the one or more light sources 206 can emit light 221, which is delivered to the substrate 202 (e.g., via the stem 204), and the substrate 202 can direct the light 221 to the eyelids 220, 222 (e.g., at the outer surface of the respective eyelid, at the outer margins of each respective eyelid, etc.), in which the light 221 causes the therapeutic effect in the eyelid tissue. As another example, the one or more light sources 206 can emit light 221, which is delivered to the substrate 202, and the substrate 202 can emit second light, via photoluminescence of a photoluminescent material of the substrate 202 (or via fluorescence of a fluorescent material of the substrate 202), towards the eyelids 220, 222, in which the second light causes the therapeutic effect in the eyelid tissue.

In some embodiments, therapeutic light delivered to either or both eyelids 220, 222 (e.g., an outer margin thereof at a free end of the respective eyelid) can be a fractional illumination pattern, which can create a distribution of thermally damaged zones of tissue, which can be ablative or non-ablative. In this case, for example, the one or more light sources 106 can be a fractional laser that can create the fractional illumination pattern. In some cases, the substrate 102 and more specifically the outer convex surface 114 of the substrate 102 can direct the fractional illumination pattern at the outer surface of the eyelid. In some cases, the fractional illumination pattern can be directed at a margin (e.g., a free end) of each eyelid 220, 222. In some cases, the fractional illumination pattern can shrink the Meibomian glands of the eyelids 220, 222, or can decrease production of sebum of the Meibomian glands of the eyelids 220, 222. In this way, the fractional illumination pattern can treat Blepharitis caused by over productive, blocked, etc., Meibomian glands.

In other configurations, the therapeutic light delivered to either or both of the eyelids 220, 222 can be selectively absorbed by sebum. For example, the therapeutic light can include a wavelength of light that is the peak absorbance wavelength of sebum (e.g., for wavelengths between substantially 100 nm and 2000 nm). In some cases, the therapeutic light can include a wavelength that is substantially 1726 nm (e.g., a wavelength selective for sebum). In this way, the therapeutic light can vaporize the sebum thereby unblocking the Meibomian glands (e.g., to alleviate Blepharitis), can destroy Meibomian glands, or can shrink the Meibomian glands.

In some non-limiting examples, the therapeutic effect can be reducing inflammation of an eyelid (e.g., improving Blepharitis of the eyelid), reducing inflammation of an eye (e.g., from reducing inflammation of the eyelid), destroying microbes (e.g., pathogens) of the eyelid (e.g., destroying bacteria), improving a healing response of the eyelid (e.g., by heating the eyelid), heating the eyelid (e.g., thereby improving an immune response of the eyelid tissue), improving the blood flow of the eyelid tissue (e.g., by heating the tissue, such as from the light 221), decreasing the pain of the eyelid tissue (e.g., by heating the tissue, such as from the light 22), improving an immune response of the eyelid tissue (e.g., by heating the tissue, such as from the light 221), causing photobiomodulation of the eyelid tissue, etc. Thus, in some configurations, the light 221 can include multiple wavelengths of light, each of which when delivered to the eyelid tissue elicits a different therapeutic effect in the eyelid tissue. In some configurations, the light 221 can include light having a wavelength that elicits multiple therapeutic responses in the eyelid tissue (e.g., near infrared light can decrease inflammation or calm the immune system of the eyelid tissue through photobiomodulation of the eyelid tissue and can heat the eyelid tissue).

In some non-limiting examples, each light source 206 can emit light having a different wavelength each eliciting a different therapeutic response in the eyelid tissue. In some configurations, the light source 206 can emit light having multiple different wavelengths. For example, the light source 206 can emit the light 221 that includes blue light, red light, near infrared light, etc. In some cases, the light 221 can include other wavelengths (or other light components) that are extraneous that do not necessarily elicit a therapeutic effect in the eyelid tissue (e.g., when the light 221 is white light). In other cases, the light 221 only includes wavelengths that each elicit a therapeutic effect in the eyelid tissue. For example, the light source 206 can include a plurality of optical paths, and each optical path can include an optical component positioned within the optical path (e.g., that can alter the wavelength of the light that passes through the optical component), such as an optical filter (e.g., a near infrared filter, a red light filter, a blue light filter, etc.). In this way, one light source can be used to deliver multiple different wavelengths, and the wavelengths can be within a tight wavelength range dictated by the optical filter. As another example, a first light source 206 can deliver first light (e.g., of the light 221) having a first wavelength of light that elicits a first therapeutic effect in the eyelid tissue, a second light source 206 can deliver second light having a second wavelength of light that elicits a second therapeutic effect (different from the first therapeutic effect) in the eyelid tissue, and so on. In this configuration, with each light source tailored to a specific wavelength of light (or a wavelength range or band of light), other light parameters may be more easily adjusted (e.g., the light flux or intensity of the light emitted by the light source).

In some non-limiting examples, the light 221 (or the light delivered to the eyelid tissue, such as when the substrate 202 includes a photoluminescent material) can include violet light having a wavelength within a wavelength range of substantially (e.g., deviating by less than 10 percent from) 380 nanometers (i.e., nm) to substantially 450 nm, blue light having a wavelength within a wavelength range of substantially 450 nm to substantially 485 nm, red light having a wavelength within a wavelength range of substantially 625 nm to substantially 750 nm, near infrared light having a wavelength within a wavelength range of substantially 700 nm to substantially 1000 nm or substantially 1100 nm. In some cases, the light 221 (or the therapeutic light delivered to the eyelid tissue, such as when the substrate 202 includes a photoluminescent material) can include light (i.e., violet light or blue light) having a wavelength within a wavelength range of substantially 400 nm to 470 nm, red light having a wavelength within a wavelength range of substantially 620 nm to substantially 750 nm, infrared light (e.g., near infrared light) having a wavelength within a wavelength range of substantially 800 nm to substantially 1100 nm. In some cases, the light 121 (or therapeutic light) can have a wavelength of substantially 1064 nm (e.g., corresponding to photobiomodulation). In some configurations, the light 121 (or therapeutic light) can be ultraviolet light (e.g., UVA light, UVB light, etc.). For example, the light 121 (or the therapeutic light) can be within a wavelength range of substantially 100 nm to substantially 400 nm, in a wavelength range of substantially 315 nm to substantially 400 nm (e.g., corresponding to UVA light), in a wavelength range of substantially 280 nm to substantially 315 nm, etc. In some configurations, UV light that is the therapeutic light applied to the eyelid(s) 120, 122 can be advantageous in that the UV light can kill pathogens (e.g., including those that can be causing the Blepharitis), and the UV light can decrease inflammation (e.g., by calming the immune system) for conditions including eczema and psoriasis each of which can cause or worsen Blepharitis. Although the wavelength ranges have been described as being substantially within the number ranges, it is appreciated that the wavelength ranges can be exact ranges (e.g., substantially 380 nm to substantially 450 nm can be 380-450 nm, 342 nm to 450 nm, etc.).

In some non-limiting examples, when the light 221 (or light delivered to the eyelid tissue) includes multiple different wavelengths (with each eliciting a different therapeutic effect), the subcomponents of the light 221 (e.g., that has a different wavelength) can be emitted simultaneously or sequentially (e.g., iteratively). For example, the blue light of the light 221 (or light delivered to the eyelid tissue) can be emitted by the one or more light sources 206 and delivered to the eyelid tissue at a first time, and the near infrared light of the light 221 (or the light delivered to the eyelid tissue) can be emitted by the one or more light sources 206 and delivered to the eyelid tissue at a second time, which can overlap with the first time (e.g., corresponding to simultaneously) or can be separate from the first time (e.g., corresponding to sequentially).

In some non-limiting examples, the eyelid treatment system 200 can include the power source 208, which can supply power to some or all of the specific components of the eyelid treatment system 200. For example, the power source 208 can be an electrical power source (e.g., an electrical storage device, a battery, a power cord, etc.) and can provide power to the one or more light source 206, the computing device 210, etc. In some non-limiting examples, the power source 208 can be coupled to the eye device 201 (e.g., when the one or more light sources 206 are coupled to the substrate 202), which can be advantageous in that the spatial footprint of the eye device 201 can be greatly reduced) For example, the power source 208 can be coupled to the stem 204 (e.g., an end of the stem 204 positioned away from the substrate 202 (e.g., where the power source 208 is positioned further away from the eyeball 216 than the substrate 202, when the substrate 202 is placed on the eyeball 216). In this way, the power source 208, which may not be biocompatible, does not undesirably interfere with the eyeball 216 or eyelid tissue. In some cases, the power source 208 can be coupled to the substrate 202 (e.g., at the outer convex surface 214). In some cases, including when the power source 208 is not biocompatible, the power source 208 can be encapsulated (e.g., with an epoxy, polymer, hydrogel, etc.) so as to avoid direct contact with tissue and the power source 208. In some configurations, the power source 208 can be an electrical storage device, such as a battery. More specifically, the battery can be a coin cell battery, which can be fairly small (as compared to other batteries).

In some non-limiting examples, the eyelid treatment system 200 can include the one or more computing devices 210, each of which can implement some or all of the processes (or tasks) of the methods described herein, as applicable. In particular, the computing device 210 can cause each of the components of the eyelid treatment system 200 to implement a particular task (e.g., by sending instructions to a respective component). For example, the computing device 210 can cause the one or more light sources 206 to emit the light 221 to treat the eyelid tissue (e.g., the light 221 itself treating the eyelid tissue), or the light 221 causing the photoluminescent material of the substrate 202 to emit light towards the eyelid tissue that treats the eye lid tissue. Although the description of the computing device 210 refers to a computing device, this description is applicable to all of the computing devices of the eyelid treatment system 200 when implemented with more than one computing device. The computing device 210 can include typical computing components, such as, a processor device, memory, communication systems, a display, inputs (e.g., a mouse, a keyboard, a touch screen, sensors, and the like), power sources, and the like. In some cases, the computing device 210 can take on a variety of specific forms including a desktop, a laptop, a mobile device (e.g., a tablet, or a smartphone), and the like. For example, in some cases, the computing device 210 can be positioned external to the eye device 201, or can be coupled to the eye device 201 (e.g., when the power source and one or more light sources are coupled to the substrate 202).

In some non-limiting examples, the parameters of the light 221 (and light components thereof) can be changed, such as, for example, by the computing device 210. For example, the light flux, intensity, duration, etc., of the light components from each of the one or more light sources 206 can be changed (e.g., by the computing device 210), such as depending on the severity of the eyelid inflammation, chronic evidence of eyelid inflammation, etc.

In some non-limiting examples, the computing device 210 can include a processor device, memory, communication systems, and the like, so as to communicate with another computing device (e.g., a desktop computer), not shown in FIG. 4. In other cases, the computing device 210 can simply be implemented as a processor. In some non-limiting examples, another computing device (e.g., a smartphone) of the eyelid treatment system 100 (not shown) can cause the computing device 210 of the eyelid treatment system 200 to implement some or all of the steps of the processes described herein, as applicable.

As shown in FIG. 4, the eye device 201 can be placed over the eyeball 216 of a subject and over the eyelids 220, 222 (or eyelid) of the subject prior to treatment of the eyelid or eyelids 220, 222 (e.g., when one or both of the eyelids 220, 222 are closed). In some cases, the substrate 202 can be positioned away from the eyelids 220, 222 (or eyelid), such that the substrate 202 does not contact the eyelids 220, 222 (or eyelid), or the substrate 202 can be placed on the eyelids 220, 222 (or eyelid) to contact the eyelids 220, 222 (or eyelid) (e.g., the inner concave surface 212 can directly contact one or both eyelids 220, 222). Then, the one or more light sources 206 can deliver light 221 to the substrate 202, which directs the light 221 (or emits secondary light, such as when the substrate 202 includes a photoluminescent material) towards the eyelids 220, 222 (e.g., the outer surface of the eyelids 220, 222, the margin of one or more eyelids including the ends thereof) thereby treating the eyelids 220, 222 (e.g., by eliciting a therapeutic effect in the eyelid from the light applied thereto). In some non-limiting examples, the eyelid treatment system 200 can be used to treat anterior Blepharitis of the eyelids 220, 222 since the light directed by the substrate can better target the outer edge of the eyelids 220, 222, which can be the concentrated source of posterior Blepharitis issues.

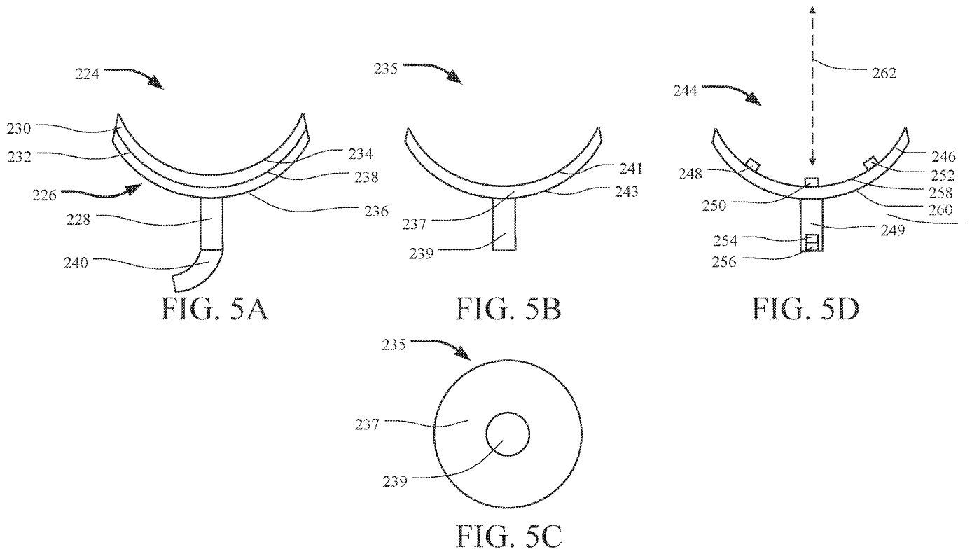

FIG. 5A shows a schematic illustration of a side view of an eye device 224, which can be implemented in a similar manner as the eye device 201. Thus, the description of the eye device 224 pertains to the eye device 201 (and vice versa). For example, the eye device 224 can include a substrate 226 and a stem 228 coupled to the substrate 226. The substrate 226 can include layers 230, 232. The layers 230, 232 can be positioned on top of each other and the layer 230 can define an inner concave surface 234, while the layer 232 can define an outer convex surface 236. The layer 232 can partially or entirely block light (e.g., ambient light) from passing therethrough to the eye. For example, the layer 232 can be formed out of an opaque material (e.g., a material with a high light absorption, such as tungsten). In this case, the opaque material can be a dark color (e.g., black) so as to absorb a large portion of the light from ambient light (e.g., environmental light), which would thus block light from passing through to the eye. In some cases, the layer 232 can be an optical filter that is configured to prevent light of a particular wavelength from passing through to the eyeball. For example, the optical filter can be configured to prevent light having the same wavelength as the therapeutic light from passing therethrough. As another example, the layer 232 can be formed out of a reflective material, which can reflect a portion (or all of the light) directed at the reflective material (e.g., ambient light away from the substrate 226 and away from the eye, or light from the one or more light sources towards the eyelid). In some cases, the reflective material can be a metal (e.g., chromium), a ceramic, etc. Unlike the layer 232, the layer 230 can be translucent or transparent so as to direct light. For example, the layer 230 can define a lens which can converge light, such as light reflected off the layer 232 (e.g., from the one or more light sources). In some configurations, and as illustrated, the layer 230 can define an inner convex surface 238 positioned above the layer 230, which can provide a reflective surface for which the ambient light can reflect off of the layer 232.

Although the layer 232 is illustrated as extending across the entire surface of the layer 230, in some configurations, the layer 232 can extend partially across the layer 230. Similarly, although the layer 230 is illustrated as extending across the entire surface of the layer 232, in some configurations, the layer 230 can extend partially across the layer 232. In this configuration, the layer 232 can extend beyond an end of the layer 230 at one end of the eye device 224 (e.g., left of the stem 228 in FIG. 5A) and can extend beyond an end of the layer 230 at another end of the eye device 224 (e.g., right of the stem 228 in FIG. 5A). In this way, the layer 232 can ensure that peripheral ambient light is prevented from reaching the eye. In some configurations, each end of the substrate 226 can include a reflective material. For example, a first end of the substrate 226 (e.g., the left side of FIG. 5A) can include a reflective material and a second end of the substrate 226 (e.g., the right side of FIG. 5A) can include a reflective material. In this way, light that would otherwise be emitted from the ends is reflected away from the end, which can avoid ambient light from entering the eyeball or otherwise interfering with the treatment, such as when the substrate 226 is relatively small in width.

In some non-limiting examples, the stem 228 can be transparent or translucent and can be optically coupled to the substrate 226 at the layer 232. For example, the stem 228 can include glass (e.g., the stem 228 can be an optical fiber, a waveguide, etc., for light propagation). In some configurations, an optical component 240 can be coupled to the stem 228 (e.g., and in some cases can be removably coupled to the stem 228). The optical component 240 can guide light (from the light source(s)) to the stem 228 and then to the substrate 226. The optical component 240 can be implemented in different ways. For example, as illustrated, the optical component 240 can be an optical fiber. In other configurations, the optical component 240 can be a waveguide. In some non-limiting examples, the optical component 240 being coupled to the stem 228 can be advantageous in that the eye device 224 can be structured in a relatively simple manner (e.g., does not require light sources, power sources, computing devices, etc., coupled to the substrate 226 or the stem 228). In addition, in some cases, the optical component 240 being removably coupled to the stem 228 can be advantageous in that the eye device 224 can be removed, cleaned (e.g., by placing in an autoclave), and reused again without forcing components (such as light sources) that may be unable to be adequately cleaned into contact with bodily fluids. Similarly, the optical component 240 being removably coupled to one or more light sources, such as the one or more light sources can be advantageous in that, again, the one or more light sources can simply be disconnected from the optical fiber and used subsequently for a different procedure without the light sources being in direct contact with bodily fluids.

FIG. 5B shows a schematic illustration of a side view of an eye device 235, which can be implemented in a similar manner as the other eye devices disclosed herein (e.g., the eye device 101). Thus, the description of the eye device 235 pertains to the other eye devices described herein (and vice versa). The eye device 235 can include a substrate 237 and a stem 239 coupled to the substrate 237. The substrate 237 can define an inner concave surface 241 and an outer convex surface 243 opposite the inner concave surface 241. In some configurations, the substrate 237 itself can partially or entirely block light from passing therethrough to the eye. For example, the substrate 237 can have optical properties, such that light is blocked from passing through the outer convex surface 243 to the eye. As a more specific example, the substrate 237 can be formed out of a material (e.g., a polymer) and can have a thickness, such that light is totally internally reflected and thus light propagates along the thickness of the substrate 237. In some cases, one or more imperfections (e.g., divots, scratches, etc.) can be included on or into the substrate 237, such that when light that is totally internally reflected and reaches an imperfection, the light stops being totally internally reflected and radiates away (in all directions) away from the imperfection. In this way, light can be emitted out from the inner concave surface 241 and delivered towards an eyelid, thereby treating the eyelid tissue. In this example, the light entering the substrate 237 would enter at an angle that allows for total internal reflection of the light. For example, the stem 239 can be angled relative to the substrate 237, such that light entering and directed by the stem 239 along with stem enters the substrate 237 at an angle that allows for total internal reflection of the light along the substrate 237.

FIG. 5C shows a schematic illustration of a top view of the eye device 235. As shown in FIG. 5C, the substrate 237 of the eye device 235 is circular (e.g., has a circular shape when viewed from the top view, as in FIG. 5C). However, in other configurations, the substrate 237 can have other shapes. Similarly, although the stem 239 of the eye device 235 is illustrated as being cylindrical in FIG. 5C, the stem 239 can have other shapes.

FIG. 5D shows a schematic illustration of a side view of an eye device 244, which can be implemented in a similar manner as the other eye devices disclosed herein (e.g., the eye device 201). Thus, the description of the eye device 244 pertains to the other eye devices described herein (and vice versa). The eye device 244 can include a substrate 246 defining an inner concave surface 258 and an outer convex surface 260 opposite the inner concave surface 258, a stem 249 coupled to the substrate 246, light sources 248, 250, 252, a power source 254, and a computing device 256. As shown in FIG. 2D, each light source 248, 250, 252 can be coupled to the substrate 246 at an inner concave surface 258 of the substrate 246 and can emit light in a direction towards the eye (and towards an eyelid of the eye), or in other words, in a direction from the outer convex surface 260 and to the inner concave surface 258. Each light source 248, 250, 252 can be implemented in a similar manner as the light sources described herein (e.g., the one or more light sources 206) and can be distributed along the substrate 246 in different ways. For example, the light source 248 can be positioned on one side of the substrate 246, the light source 252 can be positioned on an opposing side of the substrate 246, and the light source 250 can be positioned between the light sources 248, 252. In some cases, the light source 250 can be aligned with the center of the substrate 246, which can define an optical axis 262 of the substrate 246 (e.g., when the substrate 246 is implemented as a lens), such that the optical axis 262 intersects with the center of the substrate 246. Similarly, the light source 250 can be positioned such that the light source 250 is aligned with the stem 249, or the light source 250 can be substantially parallel to a longitudinal axis of the stem 249. In some cases, the light source 250 can be coupled to the stem 249 (e.g., at a side of the stem 249). In other cases, the light source 250 can be coupled to the stem 249 at a free end of the stem 249 (e.g., opposite the end of the stem 249 coupled to the substrate 246). In this way, the light source 250 (including when the eye device 244 does not include the other light sources 248, 252), can deliver therapeutic light through the stem 249 and to the substrate 246 where the substrate 246 disperses the therapeutic light to the eyelid, such that the light sources do not impede dissemination of the therapeutic light. Regardless of the configuration, the light source 250 positioned at the center of the substrate 246 can be advantageous in that light from the light source 250 can be highly targeted to the eyelid margins (e.g., the end of the eyelids), which can be the source of eyelid inflammation or Blepharitis. Also, the light from the light source 250 can treat both eyelid margins simultaneously.

Although the eye device 244 is illustrated as having three light sources 248, 250, 252, in other configurations, the eye device 244 can include other numbers of light sources (e.g., two, four, five, etc.). FIG. 2D shows the light sources 248, 250, 252 coupled to the substrate 246 at the inner concave surface 258, however, each light source 248, 250, 252 can be coupled to the substrate 246 in a different manner. For example, each light source 248, 250, 252 can be embedded within the substrate 246, coupled to the outer convex surface 260 of the substrate 246, etc. In this case, each light source 248, 250, 252 can be positioned within or aligned with a respective aperture or recess in the substrate 246. In addition, each aperture (or recess) can be covered by a transparent or translucent material, so as to allow light through to the eyelid, but also blocking debris (e.g., dust, dirt, etc.) from negatively impacting the respective light source. In some cases, the light source positioned within a recess of a substrate can be advantageous in that the light source does not directly contact the eyelid, which could irritate the eyelid.

As shown in FIG. 5D, the eye device 244 can include the power source 254 and the computing device 256, each of which can be implemented in a similar manner as the other power sources and computing devices described herein. In some cases, the power source 254 can be coupled to the stem 249 and similarly, the computing device 256 can be coupled to the stem 249. In particular, the power source 254 and the computing device 256 can be coupled to the side of the stem 249, or can be coupled to an end of the stem 249 (e.g., opposite the end of the stem 249 coupled to the substrate 246). In other cases, the power source 254 and the computing device 256 can be embedded within the stem 249 (e.g., positioned within a recess of the stem 249). Regardless of the configuration, the power source 254 (and the computing device 256) coupled to the stem 249 can be advantageous in that these components are positioned so that they do not block light from reaching an eyelid (e.g., as opposed to being coupled to the substrate 246) and the stem 249 is thicker and larger than the substrate 246, such that the stem 249 provides a larger spatial footprint for these components. In some cases, the light source 248, 250, 252 can be electrically connected to the power source 254 and the computing device 256 (e.g., via one or more wires, which can be embedded within the substrate 246), and the power source 254 can be electrically connected to the computing device 256 (e.g., via one or more wires, which can be embedded within the stem 249). In some cases, the central positioning of the power source 254 and the computing device 256 can be advantageous in that due to the central location relative to the light sources 248, 250, 252, the length of the wires are advantageously shorter than other positional relationships.

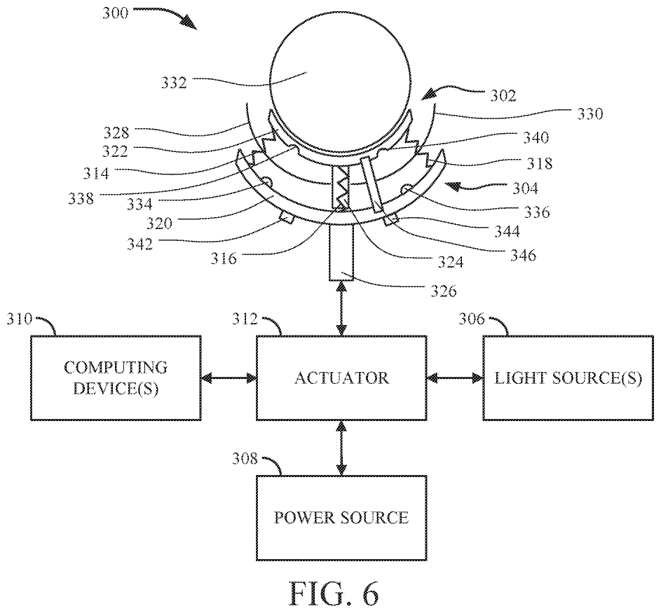

FIG. 6 shows a schematic illustration of a top, cross-sectional view of an eyelid treatment system 300, which can be implemented in a similar manner as the other eyelid treatment systems described herein (e.g., the eyelid treatment system 100). Thus, the description of the eyelid treatment system 300 pertains to the other eyelid treatment systems described herein (and vice versa). The eyelid treatment system 300 can include eye devices 302, 304, one or more light sources 306, a power source 308, one or more computing devices 310, and an actuator 312. The eye device 302 can be implemented in a similar manner as the eye devices 101, 124, 135, 144, 171, and others that deliver light to an inner surface of an eyelid, while the eye device 304 can be implemented in a similar manner as the eye devices 201, 224, 235, 224, and others that deliver light to an outer surface of the eyelid. Thus, the description of the eye devices 302, 304 pertain to the other eye devices described herein (and vice versa), as applicable. Similarly, the one or more light sources 306, the power source 308, and the computing device 310 can be implemented in a similar manner as the other corresponding devices described herein. Thus, the description of the light sources 306, the power source 308, the computing device 310, pertain to the other corresponding components described herein (and vice versa).

As shown in FIG. 6, the eyelid treatment system 100 can include springs 314, 316, 318 each of which can be coupled between the eye devices 302, 304 (e.g., between a substrate 320 of the eye device 302, and a substrate 322 of the eye device 304). The springs 314, 316, 318 can be distributed between the eye devices 302, 304 in different manners. For example, the spring 314 can be positioned to one side of the eye devices 302, 304 (e.g., the left side as shown in FIG. 6), the spring 318 can be positioned on another side of the eye devices 302, 304 (e.g., the right side as shown in FIG. 6), and the spring 316 can be positioned between the springs 314, 318 at the center of the eye device 302, 304 (e.g., at the center of the substrates 320, 322). The springs 314, 316, 318 can facilitate movement of the eye devices 302, 304 away from each other (e.g., after the substrates 320, 322 are forced closer together, for example, by the actuator 312). In other words, each spring 314, 316, 318 can be a retraction spring that can, when mechanically loaded, move the substrates 320, 322 away from each other. Although three springs 314, 316, 318 are shown in FIG. 6, the eyelid treatment system 100 can included other numbers of springs as appropriate (e.g., one, two, four, five, etc.).

In some non-limiting examples, each eye device 302, 304 (e.g., each substrate 320, 322) can include a magnet (e.g., an electromagnet) that can attract each other. In some cases, a magnet (e.g., an electromagnet) can be selectively activated (e.g. by the one or more computing devices 310). In this way, the eye devices 302, 304 (e.g., the substrates 320, 322) can be selectively attracted to expel the Meibomian glands (e.g., by cycling the magnet on an off a number of times). In some cases, a permanent magnet can be coupled to the substrate 322 (or the stem 324) of the eye device 302 and an electromagnet can be coupled to the substrate 320 (or the stem 326) of the eye device 304. In this way, the electromagnet can be energized (e.g., by the one or more computing devices 310) to cause the eye devices 302, 304 to attract together (e.g., with the eyelids 328, 330 positioned therein). Then, the electromagnet can be deenergized (or energized with current flowing in the opposing direction to reverse the magnetic field direction) to move (e.g., repel) the eye devices 302, 304 away from each other. For example, the current flowing through the electromagnet can be decreased to decrease the attractive force between the electromagnet and the magnet thereby decreasing the attractive force between them and thus loosen the compression between the eye devices 302, 304 (e.g., without inverting the magnetic field of the electromagnet). This process of attracting and repelling (or reducing attraction) can be repeated for a number of times. In some configurations, when the eye devices 302, 304 are magnetically attracted together, this can function as a lock so as to prevent the eye devices 302, 304 from moving away from each other while therapeutic light is delivered, which could direct light at undesirable regions of the subject.

In some configurations, the eye devices 302, 304 can include respective stems 324, 326, and the stems 324, 326 can be configured so as not to block movement (e.g., translation) of the eye devices 302, 304. For example, the stems 324, 326 can be offset from each other (e.g., out of alignment with each other) when, for example, the substrate 320 is placed over the eyelids 328, 330 of a subject, and the substrate 322 is placed on the eyeball 332 of the subject. In this case and others, the stems 324, 326 can be substantially parallel to each other (e.g., the longitudinal axis of each stem 324, 326 can be parallel to each other). As another example, the stem 324 of the eye device 302 can translate through or into the eye device 304 (e.g., when the eye device 304. In a first case, the stem 326 can have a hole (or bore) directed entirely therethrough (which can be aligned with a corresponding hole of the substrate 320 directed entirely therethrough). In this way, the stem 324 can pass through the hole of the substrate 320 and the hole of the stem 326, which can avoid the substrate 320 (or the stem 326) from blocking advancement of the stem 324. In a second case, the stem 326 can have a recess (which can be aligned with a corresponding hole of the substrate 320 directed entirely therethrough), such that the stem 324 can pass through the hole of the substrate 320 and into the recess of the stem 324 to avoid the substrate 320 (or the stem 326) from blocking the advancement of the stem 324. In a third case, including when the stems 324, 326 are offset from each other (or the eye device 304 does not include the stem 326), the substrate 320 can have a hole directed entirely therethrough and aligned with the stem 324, such that the stem 324 can pass through the hole of the substrate 320 to avoid the substrate 320 blocking the advancement of the stem 324. In these ways, the eye devices 302, 304 can be coupled together and can be removably coupled to each other.

In some non-limiting examples, the eyelid treatment system 300 can include the actuator 312, which can be configured to move (e.g., translate) the eye device 304 towards (and away from) the eye device 302 (and vice versa). For example, the actuator 312 can include a motor and an extender (e.g., a lead screw) that moves the extender in a direction away from the motor or in a direction back towards the motor. In this case, the extender of the actuator 312 can be coupled to the eye device 304 (e.g., at the stem 326, which can provide more rigidity for movement, or the substrate 320) and the actuator 312 can move the eye device 304 towards the eye device 302, which as described below, can compress the eyelids 328, 330 to cause Meibomian glands to excrete or expel contents blocking the gland, which could be causing the Blepharitis. Thus, the actuator 312 can push the eye device 304 towards the eye device 302. As another example, the extender of the actuator 312 can be coupled to the eye device 302 (e.g., at the stem 324, which can provide more rigidity for movement, or the substrate 322) and the actuator 312 can move the eye device 302 towards the eye device 304. Thus, the actuator 312 can pull the eye device 302 towards the eye device 304. In some cases, this pulling configuration can be advantageous in that the eyelids 328, 330 can still be compressed without compressing the eyeball 332, which could occur when the eye device 304 is pushed towards the eye device 302. As yet another example, the eye devices 302, 304 can slide past each other with either or both of the eyelids 328, 330 positioned between the eye devices 302, 304 to expel the contents of the Meibomian glands. For example, the extender of the actuator 312 can be coupled to the eye device 304 (or the eye device 302) to slide the eye device 304 over the eye device 302 (or vice versa). In this way, the sliding of the eye devices 302, 304 can purge and expel contents of the Meibomian glands of the eyelids 328, 330. In some configurations, the actuator 312 can be coupled to both eye devices 302, 304. For example, the extender of the actuator 312 can be coupled to the eye device 304, while a housing of the actuator 312 (e.g., that can contain the motor) can be coupled to the eye device 302. Similarly, the extender of the actuator 312 can be coupled to the eye device 302, while the housing of the actuator 312 can be coupled to the eye device 304. Regardless of the configuration, the actuator 312 can cause the eye devices 302, 304 to compress to squeeze the Meibomian glands, and can then cause the eye devices 302, 304 to move away from each other, with this process repeating a number of times.

In some non-limiting examples, while the eyelid treatment system 300 is shown as having the actuator 312, the eyelid treatment system 300 can include more than one actuator, which can be implemented in a similar manner as the actuator 312. The actuator 312 can be implemented in different ways. For example, the actuator 312 can be a linear actuator (e.g., an electrical linear actuator, a pneumatic linear actuator, a hydraulic linear actuator, etc.) that can include an extender and a motor, a rotational actuator (e.g., a motor that drives rotation of a component) that can cause the eye devices 302, 304 to rotate (or rock) relative to each other to expel the gland contents, etc. In some non-limiting examples, the eye device 302 can be selectively attracted (e.g., by the one or more computing devices 310) to the eye device 304, and vice versa. For example, the actuator 312 can be coupled to ether eye device 302, 304 (e.g., at a substrate thereof) and can be a magnet (e.g., an electromagnet) that can be energized (e.g., by diverting power from the power source 308 to the magnet) thereby causing the eye devices 302, 304 to attract with either or both of the eyelids 328, 330 positioned therein to expel the contents of the Meibomian glands.

As shown in FIG. 6, the eyelid treatment system 300 can include one or more protrusions or one or more recesses to help expel the contents of the Meibomian glands (e.g., which can be clogged). For example, the substrate 320 can include protrusions 334, 336, which can extend away from the substrate 320 (and the stem 326) and towards the eyeball 332. As shown in FIG. 6, the protrusions 334, 336 are positioned on opposing sides of the substrate 320, however, in other configurations, the protrusions 334, 336 can be positioned in different ways. For example, each protrusion 334, 336 can be positioned along a center of the substrate 320 (or proximate the center of the substrate 320). In this case, the protrusions 334, 336 can be located such that the protrusions 334, 336 are brought into contact with a margin of the eyelid, which has a higher likelihood of containing or a higher density of Meibomian glands to expel them. In some configurations, the substrate 322 can include recesses 338, 340, which can be aligned with the respective protrusions 334, 336 (e.g., when the eye devices 302, 304 are placed on the subject). In this way, when the eye devices 302, 304 are brought closer together, the protrusion 334 contacts the eyelid 328 to force the eyelid 328 into the recess 338, and the protrusion 334 contacts the eyelid 330 to force the eyelid 330 into the recess 340. In this way, when the eyelid is forced into the recess, the Meibomian glands may be better expelled.

Although the substrate 320 is illustrated as having the protrusions 334, 336, and the substrate 322 is illustrated as having the recesses 338, 340, in other configurations, the substrate 320 can include the recesses 338, 340 and the substrate 322 can include the protrusions 334, 336. Similarly, in some non-limiting examples, the substrate 322 can have one or more protrusions (e.g., the recesses 338, 340 can be replaced with protrusions). In this way, one or more protrusions one the substrate 320 can be aligned with open or more protrusions on the substrate 322, which can provide a high pressure zone on an eyelid when the eyelid is positioned therebetween to better expel the Meibomian glands. Although the eyelid treatment system 300 is illustrated as having two protrusions and two recesses, the eyelid treatment system 300 can include other numbers of protrusions and recesses. For example, the substrate 320 can include a number (e.g., one, three, five, twenty, fifty, etc.) of protrusions, recesses, or both. Similarly, the substrate 322 can include a number (e.g., one, three, five, twenty, fifty, etc.) of protrusions, recesses, or both. As shown in FIG. 6, the protrusions 334, 336 correspond in shape to the respective recesses 338, 340, however, in other configurations, the protrusions 334, 336 do not have the same shape as the recesses 338, 340 (and vice versa). Similarly, the protrusions 334, 336 are illustrated as being semi-circular or hemispherical, which can be advantageous to avoid inadvertent damage to an eyelid, the protrusions can have other shapes (e.g., pyramidal, a prism, etc.).

In some non-limiting examples, the eyelid treatment system 300 can vibrate the eye device 302, the eye device 304, or both, which can aid in expelling the potentially blocked Meibomian glands. For example, the eyelid treatment system 300 can include one or more vibrators that can vibrate the 320 against the eyelids 328, 330, the substrate 322 against the eyelids 328, 330, or both. As a more specific example, the eye device 304 can include vibrators 342, 344, which can be coupled to the substrate 320 (e.g., at the outer convex surface of the substrate 320). The vibrator 342, 344 can be implemented in different ways. For example, each vibrator 342, 344 can be electrically connected to the power source 308 and can be a transducer (e.g., piezoelectric transducer). Regardless, each vibrator 342, 344 can vibrate the substrate 320 thereby vibrating the eyelids 328, 330 to expel contents from the Meibomian glands of the eyelids 328, 330. Although the eyelid treatment system 300 shows two vibrators 342, 344, the eyelid treatment system 300 can include other numbers of vibrators (e.g., one, three, four, etc.) coupled to different portions of the eyelid treatment system 300. For example, a vibrator can be coupled to the eye device 302 (e.g., at the stem 324), can be coupled to the eye device 304 (e.g., at the stem 326). In some cases, a vibrator is not coupled to either eye device 302, 304, but is rather brought into contact with an eye device 302, 304 to vibrate that eye device 302, 304.

In some non-limiting examples, the one or more light sources 306 can deliver light to both eye devices 302, 304 (e.g., simultaneously, or sequentially) to deliver light to either or both eyelids 328, 330. In other cases, a first light source of the one or more light sources 306 can be coupled to the substrate 320 and a second light source of the one or more light sources 306 can be coupled to the substrate 322. Regardless of the configuration, the power source 308 can provide power to the one or more light sources 306. Similarly, the power source 308 can provide power to the actuator 312 (e.g., to extend and retract the extender of the actuator 312) and the one or more computing devices 310. In some non-limiting examples, both substrates 320, 322 having a corresponding reflective surface can be advantageous. For example, light emitted by the one or more light sources 306 and delivered to either or both of the eye devices 302, 304 can be emitted towards an eyelid (e.g., the eyelid 330) with some of the light passing through the eyelid. The light passing through the eyelid can reflect off the substrate 320 and can be directed back towards the eyelid, where a subsequent portion of the light passes through the eyelid. This subsequent portion of the light reflects off the substrate 322 and is directed back towards the eyelid. This process can be repeated so on and so forth and can be advantageous in that the amount of light required (and thus power required) can be substantially less, which can not only prevent waste of light, but can highly target light to just the eyelid(s).

In some non-limiting examples, the eyelid treatment system 300 can include a lock 346, which can prevent movement of the eye devices 302, 304 when the lock is locked. In addition, when the lock 346 is unlocked, the eye devices 302, 304 can be free to move away from each other. As shown in FIG. 6, the lock 346 can be coupled between the substrates 320, 322. However, in other configurations, the lock 346 can be coupled between the stems 324, 326 (or a stem and a substrate). The lock can be implemented in different ways. For example, the lock 346 can be a clamp that clamps the components together. As another example, the lock 346 can include a bolt and a nut, in which the nut can be threadingly engaged with the bolt to adjust the distance between the eye devices 302, 304. The lock 346 can be advantageous in that the lock 346 can ensure that the substrates 320, 322 are kept in place during delivery of therapeutic light to the eyelids to ensure that the therapeutic light is directed at the eyelids and that the therapeutic light is not directed at undesirable locations (e.g., at the practitioner).

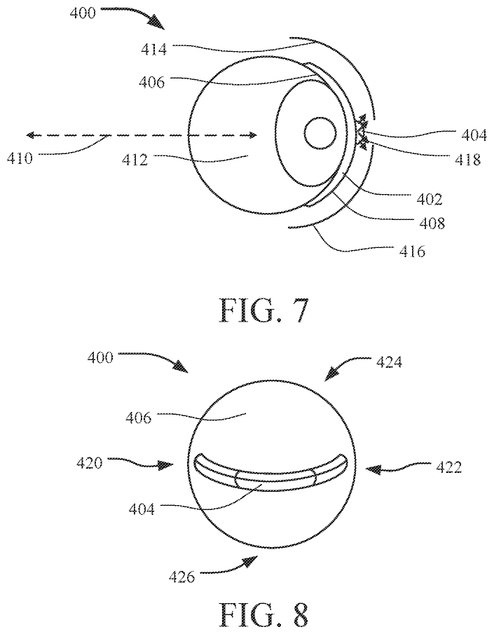

FIG. 7 shows a schematic illustration of a side view of an eye device 400, which can be implemented in a similar manner as the other eye devices disclosed herein (e.g., the eye device 101). Thus, the description of the eye device 400 pertains to the other eye devices described herein (and vice versa). The eye device 400 can include a substrate 402 and a protrusion 404 coupled to the substrate 402. The substrate 402 can have an inner concave surface 406 and an outer convex surface 408 opposite the inner concave surface 406. As shown in FIG. 7, the protrusion 404 can be coupled to the outer convex surface 408 of the substrate 402. In some configurations, the protrusion 404 can be positioned on a center of the substrate 402. For example, the protrusion 404 can be positioned, such that an optical axis 410 of the substrate 402 or an optical axis of the eyeball 412 intersects with protrusion 404. In some cases, this central location of the protrusion 404 can be desirable so as to better target the inner margins of the eyelids 414, 416. For example, therapeutic light 418 can be emitted out from the protrusion in a direction away from the eyeball 412 and towards either or both of the eyelids 414, 416 at an inner margin thereof. In some cases, the therapeutic light 418 can be emitted above the optical axis 410, and can be emitted below the optical axis 410 (e.g., to target the inner margin of both eyelids 414, 416 that could be causing the Blepharitis).

In some configurations, the optical axis 410 can simply be an axis (e.g., that bisects the substrate 402 in two), when for example, the substrate 402 is not a lens. In other configurations, a light source can be positioned underneath the protrusion 404 or can be embedded within the protrusion 404 to emit light through the protrusion 404 and to either or both eyelids 414, 416. In this case, the optical axis 410 can be an optical axis of the light source. In some cases, the protrusion 404 can be an optical component, such as a lens. For example, the protrusion 404 can be a converging lens that converges the therapeutic light. As shown in FIG. 7, when the substrate 402 is placed on the eyeball 412, the protrusion 404 can be positioned between the eyelids 414, 416 (e.g., between the free ends thereof), can be positioned below the eyelid 414, and can be positioned above the eyelid 416. Although the protrusion 404 is illustrated as being curved, and more specifically, having a hemispherical cross-section, in other configurations, the protrusion 404 can have other shapes (e.g., a prism). The protrusion 404 can be formed out of the same material as the substrate 402, or can be formed out of a different material. The protrusion 404 can be transparent or translucent so as to allow the therapeutic light therethrough. In some configurations, the protrusion 404 can be positioned on a maxima (e.g., a global maxima) of the substrate 402.

In some embodiments, the substrate 402 can include a reflective material (or an opaque material) positioned on the outer convex surface 408 (or a convex surface embedded within the substrate 402). In some cases, the reflective material can be a layer, and a portion of the reflective material can extend above the optical axis 410 of the substrate 402 and can extend below the optical axis 410 of the substrate 402. In this way, the reflective material at a convex surface of the substrate 402 can not only block light (e.g., therapeutic light) from undesirably reaching the sensitive structures of the eyeball 412, but can also redirect the light back at either or both of the eyelids 414, 416. In some cases, the reflective material (or the opaque material) can be positioned above the protrusion 404, below the protrusion 404, etc. In this way, the therapeutic light can be focused more directly at the margins of the eyelids, via the protrusion 404.

FIG. 8 shows a schematic illustration of a top view of the eye device 400. As shown in FIG. 8, the protrusion 404 can extend along the substrate 402 to a first lateral side 420 of the substrate 402, can extend past a center of the substrate 402, and can extend to a second lateral side 422 of the substrate 402 opposite the first lateral side 420. In some cases, the protrusion 404 can be a single body or can be segmented into sections along the substrate 402. In some cases, the protrusion 404 can be a ridge, a raised strip, etc. In some cases, the protrusion 404 can extend upwardly towards an upper end 424 of the substrate 402 and past the free end of the eyelid 414 (e.g., when the substrate 402 is placed on the eyeball 412). Similarly, the protrusion 404 can extend downwardly towards a lower end 426 of the substrate 402 and past the free end of the eyelid 416 (e.g., when the substrate 402 is placed on the eyeball 412). Although not shown in FIGS. 7 and 8, the eye device 400 can include a stem, which can be coupled to the same side (e.g., the outer side) of the substrate 402 as the protrusion 404. In some cases, the stem can be aligned with the protrusion 404 (e.g., the stem can overlap with the protrusion 404), or the stem can be offset from the protrusion 404.

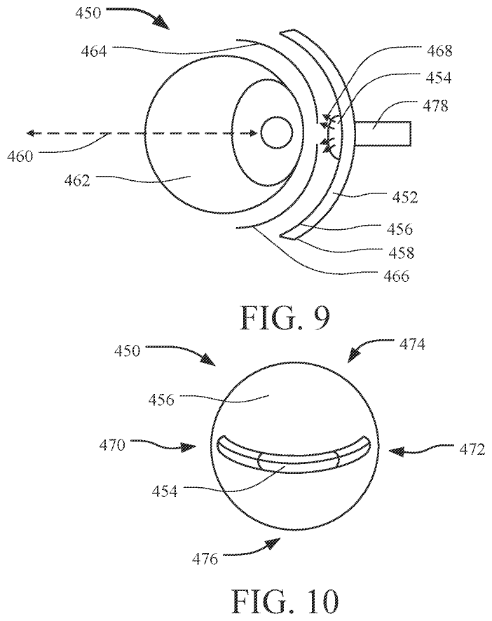

FIG. 9 shows a schematic illustration of a side view of an eye device 450, which can be implemented in a similar manner as the other eye devices disclosed herein (e.g., the eye device 101). Thus, the description of the eye device 450 pertains to the other eye devices described herein (and vice versa). The eye device 450 can include a substrate 452 and a protrusion 454 coupled to the substrate 452. The substrate 452 can have an inner concave surface 456 and an outer convex surface 458 opposite the inner concave surface 456. As shown in FIG. 7, the protrusion 454 can be coupled to the inner concave surface 456 of the substrate 408. In some configurations, the protrusion 454 can be positioned on a center of the substrate 452. For example, the protrusion 454 can be positioned, such that an optical axis 460 of the substrate 452 or an optical axis of the eyeball 462 intersects with protrusion 454. In some cases, this central location of the protrusion 454 can be desirable so as to better target the outer margins of the eyelids 464, 466. For example, therapeutic light 468 can be emitted out from the protrusion 454 in a direction towards the eyeball 412 and towards either or both of the eyelids 464, 466 at an outer margin thereof. In some cases, the therapeutic light 468 can be emitted above the optical axis 460, and can be emitted below the optical axis 460 (e.g., to target the outer margin of both eyelids 464, 466 that could be causing the Blepharitis).

In some configurations, the optical axis 460 can simply be an axis (e.g., that bisects the substrate 452 in two), when for example, the substrate 452 is not a lens. In other configurations, a light source can be positioned underneath the protrusion 454 or can be embedded within the protrusion 454 to emit light through the protrusion 454 and to either or both eyelids 464, 466. In this case, the optical axis 460 can be an optical axis of the light source. In some cases, the protrusion 454 can be an optical component, such as a lens. For example, the protrusion 454 can be a converging lens that converges the therapeutic light. As shown in FIG. 9, when the substrate 452 is placed over the eyeball 462 (and over each or both eyelids 464, 466), the protrusion 454 can be positioned between the eyelids 464, 466 (e.g., between the free ends thereof), can be positioned below the eyelid 464, and can be positioned above the eyelid 466. Although the protrusion 454 is illustrated as being curved, and more specifically, having a hemispherical cross-section, in other configurations, the protrusion 454 can have other shapes (e.g., a prism). The protrusion 454 can be formed out of the same material as the substrate 452, or can be formed out of a different material. The protrusion 454 can be transparent or translucent so as to allow the therapeutic light therethrough. In some configurations, the protrusion 454 can be positioned on a minima (e.g., a global minima) of the substrate 402. As shown in FIG. 9, the eye device 450 can include a stem 478, which can be coupled to an opposing side (e.g., the outer side) of the substrate 452 as the protrusion 454 (e.g., the protrusion 454 coupled to the inner side of the substrate 452). In some cases, the stem 478 can be aligned with the protrusion 454 (e.g., an axis such as the optical axis 460 can intersect the stem 478 and the protrusion 454), or the stem 478 can be offset from the protrusion 454.

In some embodiments, the substrate 452 can include a reflective material (or an opaque material) positioned on the inner concave surface 456 (or a concave surface embedded within the substrate 452). In some cases, the reflective material can be a layer, and a portion of the reflective material can extend above the optical axis 460 of the substrate 452 and can extend below the optical axis 460 of the substrate 402. In this way, the reflective material at a concave surface of the substrate 452 can not only block light (e.g., therapeutic light) from undesirably reaching the sensitive structures of the eyeball 462, but can also redirect the light back at either or both of the eyelids 464, 466. In some cases, the reflective material (or the opaque material) can be positioned above the protrusion 454, below the protrusion 454, etc. In this way, the therapeutic light can be focused more directly at the margins of the eyelids, via the protrusion 454.

FIG. 10 shows a schematic illustration of a top view of the eye device 450. As shown in FIG. 10, the protrusion 454 can extend along the substrate 452 to a first lateral side 470 of the substrate 452, can extend past a center of the substrate 452, and can extend to a second lateral side 472 of the substrate 452 opposite the first lateral side 470. In some cases, the protrusion 454 can be a single body or can be segmented into sections along the substrate 452. In some cases, the protrusion 454 can be a ridge, a raised strip, etc. In some cases, the protrusion 454 can extend upwardly towards an upper end 474 of the substrate 452 and past the free end of the eyelid 464 (e.g., when the substrate 452 is placed on the eyeball 462). Similarly, the protrusion 454 can extend downwardly towards a lower end 476 of the substrate 452 and past the free end of the eyelid 466 (e.g., when the substrate 452 is placed on the eyeball 462).

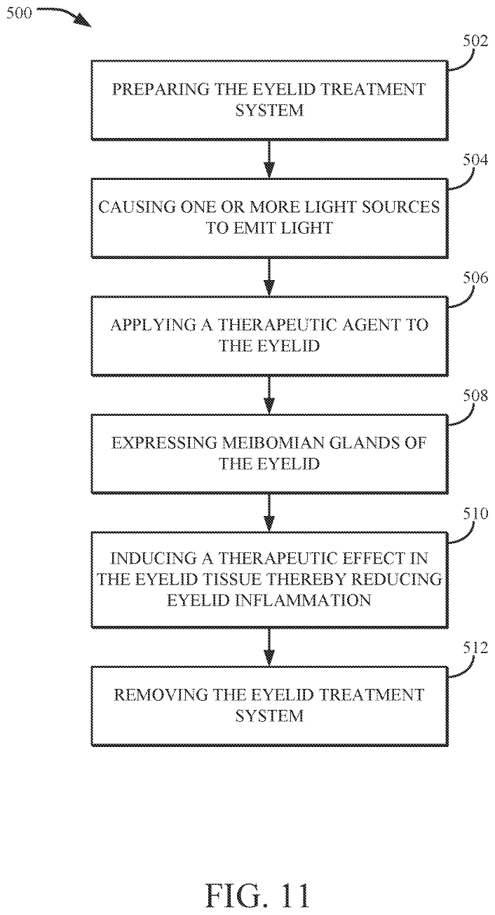

FIG. 11 shows a flowchart of a process 500 for reducing eyelid inflammation in an eyelid (e.g., or multiple eyelids) of a subject. The process 500 can be implemented using any of the systems described herein, as appropriate. For example, the process 500 can be implemented using the eyelid treatment system 100, 170, 200, 300. In some non-limiting examples, some or all of the blocks of the process 500 can be implemented using one or more computing devices (e.g., the one or more computing devices 110), as appropriate (e.g., where the process 500 can be a computer implemented method).

At 502, the process 500 can include preparing an eyelid treatment system (e.g., the eyelid treatment system 300), which can include placing an eye device (e.g., the eye device 302) on an eye of a subject. In some cases, this can include grasping a stem of the eye device and placing a substrate of the eye device into contact with an eyeball of the eye of the subject (e.g., after one or more eyelids of the eye are opened). The block 502 can include placing an eye device (e.g., the eye device 304) over an eyelid of the eye of the subject. This can include grasping a stem of the eye device and placing a substrate of the eye device over the eyelids (or eyelid) of the eye of the subject. In some configurations, the block 502 can include coupling one or more light sources to either or both of the eye devices via a stem of an eye device. This can include coupling the one or more light sources to either or both of the eye devices, via an optical guide (e.g., an optical fiber).

At 504, the process 500 can include causing (e.g., using one or more computing devices), one or more light sources to emit light thereby causing a therapeutic effect in the eyelid that reduces inflammation of the eyelid. In some cases, the light directed at the eyelid can cause the therapeutic effect, while in other cases, the light can be a first light that excites either or both substrates to emit second light at the eyelid, where the second light induces the therapeutic effect in the eyelid. Regardless of the configuration, the therapeutic light from the one or more light sources (or caused by the one or more light sources) can be delivered to an inner surface of the eyelid (e.g., via the substrate placed on the eye) can be delivered to an outer surface of the eyelid (e.g., via the substrate placed over the eyelid), delivered to a margin of the eyelid (e.g., the inner surface of the margin, the outer surface of the margin, etc.). In some cases, the therapeutic light delivered to the eyelid (e.g., the inner surface of the eyelid, the outer surface of the eyelid, or both), can include multiple different wavelengths of light each of which can cause a different therapeutic effect in the eyelid that reduces (or that could reduce) eyelid inflammation. In this way, multiple different wavelengths of light can treat Blepharitis where the exact cause of the Blepharitis could be unknown.

At 506, the process 500 can include applying a therapeutic agent to the eyelid. This can include manually applying (e.g., via a practitioner, such as a doctor), a topical therapeutic agent (e.g., described above) to the eyelid (e.g., at an outer surface of the eyelid, an inner surface of the eyelid, etc.). In some cases, this can include applying a drop containing the therapeutic agent to the eyeball, such that after the therapeutic agent is delivered to the eye, the therapeutic agent migrates to the eyelid. This can also include the therapeutic agent migrating from the substrate and into contact with the eyelid (e.g., at an inner surface of the eyelid, at an outer surface of the eyelid, etc.).

At 508, the process 500 can include expressing Meibomian glands of the eyelid. In some cases, this can include compressing the eyelid between the substrates of the eye devices (e.g., the substrates 320, 322 of the eye devices 304, 302). In some cases, this can include causing (e.g., using one or more computing devices) an actuator to move the substrates together to express the contents of the Meibomian glands. In some cases, this can include causing an actuator to slide the substrates across each other to express the Meibomian glands of the eyelid (e.g., to compress the eyelid to express the glands). In some cases, expressing the Meibomian glands can include unblocking the Meibomian glands.

At the block 510, the process 500 can include reducing inflammation of the eyelid. The block 510 can include inducing a therapeutic effect in the eyelid tissue thereby reducing the eyelid inflammation. In some cases, inducing a therapeutic effect in the eyelid tissue can be caused by the therapeutic light applied to the eyelid, the therapeutic agent in contact with the eyelid, the expressing of the Meibomian glands, etc. In some cases, the block 510 can include inducing a plurality of different therapeutic effects in the eyelid tissue thereby reducing the eyelid inflammation. For example, the eyelid inflammation can be reduced by two different mechanisms applied to the eyelid (e.g., the therapeutic light and expressing of the Meibomian glands).

After the block 510, or at other points in the flowchart, the process 500 can proceed back to the block 502. For example, after one eyelid is treated, the other eyelid of the same eye can be treated. In some cases, this can include rotating either or both of the substrates (e.g., about a stem of the substrate) until each substrate covers the next eyelid to be treated.

At 512, the process 500 can include removing the eyelid treatment system. For example, this can include decoupling the one or more light sources from each eye device (e.g., at the stem of the eye device). This can include removing the eye device that is positioned over the eyelid(s) and the eye (e.g., by grasping the stem). Similarly, including after the eye device that is over the eyelids is removed, this can include removing the eye device in contact with the eyeball. In some cases, after the substrates have been removed, each substrate can be cleaned, disinfected, etc. This can include placing the eye devices in an autoclave, such that the eye devices can be reused for a different patient.

In some non-limiting examples, after the block 512, the process 500 can proceed back to the block 502 to, for example, treat the other eyelid(s) of the other eye, as applicable. The above description for treating an eyelid of one eye can be used for treating the eyelid(s) of the opposing eye.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

Some non-limiting examples of the disclosure provide an optical contact lens for the treatment of Blepharitis.

Disclosed herein is a new medical device, an optical contact lens, for the treatment of blepharitis, which is inflammation of the eyelids. Blepharitis can be acute or chronic; chronic blepharitis is the more common form and is estimated to affect as many as 25 million Americans. A variety of treatments are used, including topical antibiotics, topical steroids, thermal therapy of the eyelids (heating), and milking of the Meibomian glands (expression of contents of the glands). None of these treatments has been shown effective in clinical studies. As described herein, a novel “optical contact lens” is proposed to address this common yet unsolved clinical problem.

In certain non-limiting examples, the disclosure comprises a contact lens-like device made from bio compatible materials (e.g., hydrogels, silicone, etc.) that can emit light from the lens surface. The device may be comprised of either one or two “Optical Contact Lenses”. One lens (inner lens) may be placed on the surface of the globe (eye ball) inside the eye. The inner lens may be opaque at the concave surface to prevent the light exposure to the cornea and retina and light is emitted from the convex surface to the inner eye lid. The other lens (outer lens) may be placed on the outer eye lid. In contrast to the inner lens, the outer lens may be opaque at convex surface (to avoid light induced environmental hazard) and light is emitted from the concave surface to the outer eye lid. Multiple wavelengths can be delivered via the Optical Contact Lens, including violet/blue light (400-470 nm) for antibacterial effect, red light (620-750 nm) or near infrared irradiation (800-1100 nm) for photobiomodulation and for heating of tissue.

The “Optical Contact Lens” can also be used in combination of topical antibiotic drops and/or topical steroids, the current standard of care for blepharitis, for potential synergistic therapeutic effect. These other medications can be applied separately from the contact lens, or can be incorporated into the contact lens and released by the contact lens.

The contact lens can also include a compressive component, in which the device compresses the eyelid margin in an intermittent fashion in order to release debris from the glands on the inner eyelid surface (milking of the glands).

Some non-limiting examples of the disclosure are described herein with reference to the accompanying figures. The description, together with the figures, make apparent to a person having ordinary skill in the art how some non-limiting examples of the disclosure can be practiced. The figures are for the purpose of illustrative discussion and no attempt is made to show structural details of an non-limiting example in more detail than is necessary for a fundamental understanding of the teachings of the disclosure.

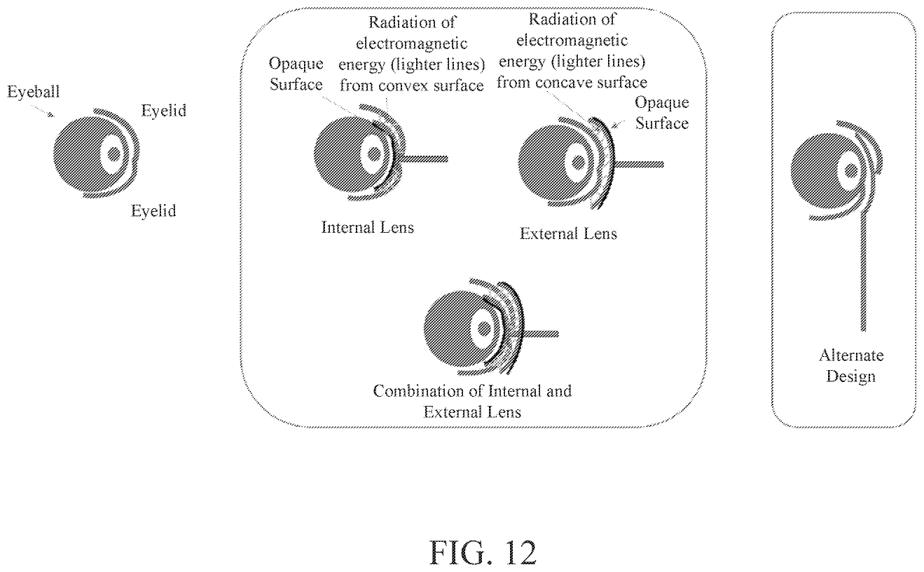

FIG. 12 shows a schematic of various optical contact lenses in accordance with an non-limiting example of the disclosure.

A preferred non-limiting example of the disclosure can comprise a contact lens-like device made from bio-compatible transparent or translucent materials (e.g., hydrogels, silicone rubber or other silicone polymers, acrylates, etc.) that can deliver light from the lens surface. The material can also be fluorescent, capable of converting an excitation wavelength to a longer, emission wavelength. The device can comprise one or two “Optical Contact Lenses.” In this non-limiting example, one lens (inner lens) can be placed on the surface of the globe (eye ball). The inner lens is opaque at its concave surface in contact with the eye, to prevent the light exposure to the cornea and retina, while light is delivered from the convex surface to the inner eye lid. The other lens (outer lens) will be placed on the outer eye lid. In contrast to the inner lens, the outer lens is opaque at convex surface (to avoid light-induced environmental hazard) and light is emitted from the concave surface to the outer eye lid.

Multiple wavelengths can be delivered via the Optical Contact Lens, including violet/blue light (400-470 nm) for antibacterial effect, and/or red light (620-750 nm) and/or near infrared irradiation (800-1100 nm) for photobiomodulation, a process that reduces inflammation and pain. Absorption of light in the eyelid can produce mild heating, which provides further benefit by increasing blood flow and healing responses. These wavelengths can be delivered singly, simultaneously, or sequentially. The light can be pulsed or continuous. Irradiance and fluence delivered are chosen to be in a therapeutic range; irradiance is the primary determinant of tissue heating, which places an upper limit of about 300 mW/cm2 (about twice the irradiance of sunlight). The “Optical Contact Lens” can also be used in combination with topical antibiotic drops and/or topical steroids, the current standard of care for Blepharitis, for potential synergistic therapeutic effect. These other medications can be applied separately from the contact lens, or can be incorporated into the contact lens and released by the contact lens. The contact lens can also include a compressive component, in which the device compresses the eyelid margin in an intermittent fashion in order to release debris from the glands on the inner eyelid surface (milking of the glands).

The device can be composed of either one or two “Optical Contact Lens” connected with light source(s) through optical waveguides and/or fiber optics for multiple therapeutic effects on both anterior and posterior blepharitis. The product is simple, portable, reusable, can be cleaned between uses, and can be used by consumers/patients at home and/or as an in-office treatment.

The inner surface (e.g., the concave surface) of the inner lens can be constructed so as to preclude emission of light into the eye itself. This can be accomplished by a coating on its inner surface. The coating can be made of a material that absorbs any light that can be propagating toward the eye. For example, a highly absorptive black plastic or other similar material can accomplish this. The inner coating can instead be a highly reflective surface that reflects (in the opposite direction) any light that is emitted in the direction of the cornea. The latter has the advantage of limiting “wasted photons” Alternatively, the lens itself (i.e., not a coating) can be constructed so as to preclude light toward the cornea by making the lens itself highly reflective.

The light source. For the inner lens, the light source can be outside of the eye, with light transmitted to the lens via an optical guide. The lens can be constructed to emit/project this light in the preferred direction. For the inner lens, the lens itself can serve as or contain the light source by incorporation of small light sources, such as light emitting diodes, within it or on its convex surface. For the outer lens, the light source can be external to it. It can be incorporated into the lens. It can be located on its concave (inner) surface.

The inner lens can have a stem that protrudes from the convex surface. The stem can serve to simplify placement of the lens into the eye. The stem can include element(s) to bring light from outside the eye to the lens (such as optical fibers). The stem can include a light source (such as LED). The stem can include a power source which powers the light source. The stem can serve to connect the inner lens to an external lens that sits on the outer surface of the eyelid.

Compression (e.g., mechanical “milking” of the sebaceous glands) can be accomplished by applying application of pressure to the glands in order to massage them. The pressure can be applied axially (pressing external lens against inner lens or vice-versa) or through a sliding mechanism of one lens sliding relative to the other. Compressive or sliding motion can be accomplished via a spring-loaded mechanism (e.g. a spring connecting the two lenses), using an electrically powered motor or air compression in order to compress them together). Alternatively, it can be accomplished via electromagnetic attraction or repulsion. The lenses can contain small “bumps” and/or “indentations” to further enhance compression.

It will be appreciated by those skilled in the art that while the disclosed subject matter is described herein in connection with particular non-limiting examples and examples, the disclosure is not necessarily so limited, and that numerous other non-limiting examples, examples, uses, modifications and departures from the non-limiting examples, examples and uses are intended to be encompassed by the claims attached hereto. Each article cited herein is incorporated by reference in its entirety.

This disclosure describes a new medical device, Optical Contact Lens, for the treatment of blepharitis, which is inflammation of the eyelids. Blepharitis can be acute or chronic; chronic blepharitis is the more common form, and is estimated to affect as can as 25 million Americans. A variety of treatments are used, including topical antibiotics, topical steroids, thermal therapy of the eyelids (heating), and milking of the Meibomian glands (expression of contents of the glands). None of these treatments has been shown effective in clinical studies. A novel “optical contact lens” is proposed to address this common yet unsolved clinical problem. A preferred non-limiting example of the disclosure employs contact lens-like device made from bio-compatible transparent or translucent materials (e.g., hydrogels, silicone rubber or other silicone polymers, acrylates, etc.) that can deliver light from the lens surface. The material can also be fluorescent, capable of converting an excitation wavelength to a longer, emission wavelength. The device consists of either one or two “Optical Contact Lenses”. In this non-limiting example, one lens (inner lens) is placed on the surface of the globe (eye ball). The inner lens is opaque at its concave surface in contact with the eye, to prevent the light exposure to the cornea and retina, while light is delivered from the convex surface to the inner eye lid. The other lens (outer lens) will be placed on the outer eye lid. In contrast to the inner lens, the outer lens is opaque at convex surface (to avoid light-induced environmental hazard) and light is emitted from the concave surface to the outer eye lid. Multiple wavelengths can be delivered via the Optical Contact Lens, including violet/blue light (400-470 nm) for antibacterial effect, and/or red light (620-750 nm) and/or near infrared irradiation (800-1100 nm) for photobiomodulation, a process that reduces inflammation and pain. Absorption of light in the eyelid can produce mild heating, which provides further benefit by increasing blood flow and healing responses. These wavelengths can be delivered singly, simultaneously, or sequentially. The light can be pulsed or continuous. Irradiance and fluence delivered are chosen to be in a therapeutic range; irradiance is the primary determinant of tissue heating, which places an upper limit of about 300 mW/cm2 (about twice the irradiance of sunlight). The “Optical Contact Lens” can also be used in combination of topical antibiotic drops and/or topical steroids, the current standard of care for blepharitis, for potential synergistic therapeutic effect. These other medications can be applied separately from the contact lens, or can be incorporated into the contact lens and released by the contact lens. The contact lens can also include a compressive component, in which the device compresses the eyelid margin in an intermittent fashion in order to release debris from the glands on the inner eyelid surface (milking of the glands).

The Optical Contact Lens that can be made from compatible, light-transmitting and/or light-emitting materials and can deliver multiple wavelengths of therapeutic light to the (inner or/and outer) eye lids for the treatment of (anterior or/and posterior) blepharitis with no exposure to the retina and cornea.

The device itself would be commercialized. The product will be composed of either one or two “Optical Contact Lens” connected with light source(s) through optical waveguides and/or fiber optics for multiple therapeutic effects on both anterior and posterior blepharitis. The product is simple, portable, reusable, can be cleaned between uses, and can be used by consumers/patients at home and/or as an in-office treatment.

The present disclosure has described one or more preferred non-limiting examples, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single-or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A, B; C; A and B; A and C; B and C, and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Also as used herein, unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, ±10%, +5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

Various features and advantages of the disclosure are set forth in the following claims.

We claim:

1. An eyelid treatment system comprising:
a contact lens including an inner concave surface and an outer convex surface opposite the inner concave surface, the contact lens configured to be placed on an eyeball of a subject, the inner concave surface being configured to at least partially block light from being transmitted through the inner concave surface;
a light source optically coupled to the contact lens, the light source being configured to deliver light to an inner surface of an eyelid of the subject when the contact lens is placed on the eyeball; and
wherein the inner concave surface is configured to at least partially block light from being transmitted through the inner concave surface, such that the light propagates in a direction away from the eyeball of the subject
wherein the light from the light source is configured to reduce eyelid inflammation.

2. The eyelid treatment system of claim 1, wherein the inner concave surface includes an opaque material.

3. The eyelid treatment system of claim 2, wherein the inner concave surface is positioned on a first side of the contact lens and a second side of the contact lens; and
wherein an optical axis of the contact lens is positioned between the first side of the contact lens and the second side of the contact lens.

4. (canceled)

5. The eyelid treatment system of claim 1, wherein the contact lens includes an opaque material that defines the inner concave surface of the contact lens, such that the light from the light source or ambient light that is transmitted through the outer convex surface towards the inner concave surface is at least partially blocked from being transmitted therethrough; or
wherein the contact lens includes a reflective material that defines the inner concave surface of the contact lens, such that the light from the light source or the ambient light that is transmitted through the outer convex surface towards the inner concave surface is at least partially blocked from being transmitted therethrough.

6. The eyelid treatment system of claim 1, further comprising a stem coupled to the contact lens, the stem extending away from the contact lens in a direction away from the eyeball when the contact lens is placed on the eyeball.

7. The eyelid treatment system of claim 6, wherein the light source is optically coupled to the stem, such that the light from the light source is transmitted through the stem and to the contact lens.

8. The eyelid treatment system of claim 1, wherein the light from the light source has a wavelength that is:
within a first range of 400-470 nanometers, such that the light is configured to destroy microbes of the eyelid;
within a second range of 620-750 nanometers, such that the light is configured to at least one of heat eyelid tissue or to decrease inflammation or calm the immune system of the eyelid tissue through photobiomodulation; or
within a third range of 800-1100 nanometers, such that the light is configured to at least one of heat the eyelid tissue or to decrease inflammation or calm the immune system of the eyelid tissue through photobiomodulation.

9-12. (canceled)

13. The eyelid treatment system of claim 1, wherein the light from the light source is directed at, at least one of 25% of an inner surface area of the eyelid, 50% of the inner surface area of the eyelid, or 75% of the inner surface area of the eyelid.

14. The eyelid treatment system of claim 1, wherein the contact lens is a diverging lens, such that the light from the light source diverges when the light is emitted out from the outer convex surface of the contact lens.

15. An eyelid treatment system comprising:
a substrate including an inner concave surface and an outer convex surface opposite the inner concave surface, the substrate configured to be placed external to an eye of a subject, the outer concave surface being configured to at least partially block ambient light from being transmitted through the outer convex surface in a direction towards the inner concave surface;
a light source configured to deliver light to an outer surface of an eyelid of the subject when the substrate is placed over the eyelid of the subject; and
wherein the light from the light source is configured to improve eyelid inflammation.

16. The eyelid treatment system of claim 15, wherein the outer convex surface includes an opaque material.

17. The eyelid treatment system of claim 15, wherein the outer convex surface is positioned on a first side of the substrate and a second side of the substrate; and
wherein an axis of the substrate that bisects the substrate into the first side and the second side is positioned between the first side of the substrate and the second side of the substrate.

18. The eyelid treatment system of claim 15, wherein the substrate includes an opaque material that defines the outer convex surface of the substrate, such that the ambient light is at least partially blocked from being transmitted through the outer convex surface of the substrate; or
wherein the substrate includes a reflective material that defines the outer convex surface of the substrate, such that the ambient light is at least partially blocked from being transmitted through the outer convex surface of the substrate.

19. The eyelid treatment system of claim 15, further comprising a stem coupled to the substrate, the stem extending away from the substrate in a direction away from the eyelid when the substrate is placed over the eyelid.

20. (canceled)

21. The eyelid treatment system of claim 15, wherein the light from the light source has a wavelength that is:
within a first range of 400-470 nanometers, such that the light is configured to destroy microbes of the eyelid;
within a second range of 620-750 nanometers, such that the light is configured to at least one of heat the eyelid tissue or to decrease inflammation or calm the immune system of the eyelid tissue through photobiomodulation ; or
within a third range of 800-1100 nanometers, such that the light is configured to at least one of heat the eyelid tissue or to decrease inflammation or calm the immune system of the eyelid tissue through photobiomodulation.

22. (canceled)

23. The eyelid treatment system of claim 15, wherein the substrate includes a biocompatible material;
wherein the biocompatible material is at least one of a hydrogel, silicone, a polymer, or a plastic.

24. The eyelid treatment system of claim 15, wherein the light source is coupled to the substrate.

25. The eyelid treatment system of claim 15, further comprising a power source coupled to the substrate; and
wherein the light source is electrically coupled to the power source.

26. The eyelid treatment system of claim 15, wherein the light from the light source is directed at, at least one of 25% of an outer surface area of the eyelid, 50% of the outer surface area of the eyelid, or 75% of the outer surface area of the eyelid.

27. The eyelid treatment system of claim 15, wherein the substrate is a converging lens, such that when the light from the light source converges when the light is emitted out from the inner concave surface of the substrate.

28. An eyelid treatment system comprising:
a first substrate including a first inner concave surface and a first outer convex surface opposite the first inner concave surface, the first substrate configured to be placed on an eye of a subject, the first inner concave surface being configured to at least partially block light from being transmitted through the inner concave surface away from the outer convex surface;
a second substrate including a second inner concave surface and a second outer convex surface opposite the second inner concave surface, the second substrate configured to be placed external to the eye of the subject, the second outer convex surface being configured to at least partially block ambient light from being transmitted through the second outer convex surface in a direction towards the second inner concave surface;
one or more light sources configured to emit light to at least one of an inner surface or an outer surface of the eyelid of the subject when the first substrate is placed on the eye of the subject and when the second substrate is placed over the eyelid of the subject; and
wherein the light from the one or more light sources is configured to reduce eyelid inflammation.

29-37. (canceled)

38. A method of treating eyelid inflammation, the method comprising:
placing a substrate on an eye of a subject or over an eyelid of the subject;
causing one or more light sources coupled to the substrate or optically coupled to the substrate to emit first light towards the eyelid or towards the substrate; and
reducing eyelid inflammation from the first light by:
the first light emitted from the one or more light sources and directed towards the eyelid causing a therapeutic effect in the eyelid tissue that decreases eyelid inflammation of the eyelid; or
the first light emitted from the one or more light sources and directed towards the substrate causes the substrate to emit a second light different from the first light towards the eyelid, the second light causing a therapeutic effect in the eyelid tissue that decreases eyelid inflammation of the eyelid.

39. The method of claim 38, wherein the first light has a plurality of different wavelengths of light; and
wherein the first light emitted from the one or more light sources and directed towards the eyelid causes a plurality of different therapeutic effects in the eyelid tissue from the plurality of different wavelengths of light.

40. The method of claim 38, wherein the second light has a plurality of different wavelengths of light; and
wherein the second light emitted by the substrate and directed towards the eyelid causes a plurality of different therapeutic effects in the eyelid tissue from the plurality of different wavelengths of light.

41. The method of claim 38, wherein the substrate is a first substrate that is optically coupled to the one or more light sources and further comprising:
placing the first substrate on the eye of the subject;
placing a second substrate over the eyelid of the subject; and
causing the one or more light sources to deliver the first light to the first substrate and the second substrate thereby delivering the first light to opposing sides of the eyelid, the opposing sides of the eyelid being an inner side and an outer side of the eyelid.

42. The method of claim 38, wherein the substrate is a first substrate, wherein a first light source of the one or more light sources is coupled to the first substrate, wherein the therapeutic effect is a first therapeutic effect, and further comprising:
placing the first substrate on the eye of the subject;
placing a second substrate over the eyelid of the subject, the one or more light sources including a second light source coupled to the second substrate; and
causing the first light source to deliver the first light to an inner side eyelid thereby causing the first therapeutic effect in the eyelid tissue that decreases eyelid inflammation of the eyelid; and
causing the second light source to deliver third light to the eyelid thereby causing a second therapeutic effect in the eyelid tissue that decreases eyelid inflammation of the eyelid.

43. The method of claim 38, wherein the substrate includes a photoluminescent material; and
wherein the first light emitted towards the substrate excites the photoluminescent material, such that the photoluminescent material emits the second light having a wavelength longer than the first light.

44. The method of claim 38, wherein the therapeutic effect includes at least one of:
destroying microbes of the eyelid;
improving an immune response of the eyelid tissue;
improving the blood flow of the eyelid tissue; or
causing photobiomodulation of the eyelid tissue.

45. The method of claim 38, wherein the first light or the second light is blue light, violet light, red light, or near infrared light.

46. The method of claim 38, wherein the substrate is a first substrate and further comprising:
placing the first substrate on the eye of the subject;
placing a second substrate over the eyelid of the subject after placing the first substrate on the eye of the subject and after the subject closes the eyelid; and
advancing the first substrate towards the second substrate or advancing the second substrate towards the first substrate to express the Meibomian glands of the eyelid.

47-51. (canceled)
